(19) United States
(12) Patent Application Publication
(10) Pub. No.: US 2021/0275515 A1
(43) Pub. Date: Sep. 9, 2021

(54) Method of Treating Urothelial Carcinoma and Other Genitourinary Malignancies Using N-(4-(6,7-dimethoxyquinolin-4-yloxy)phenyl)-N'-(4-fluorophenyl)cyclopropane-1,1-dicarboxamide

(71) Applicant: The US of America As Represented By The Secretary, Dept. of Health and Human Services, Bethesda, MD (US)

(72) Inventor: Andrea B. Apolo, Bethesda, MD (US)

(21) Appl. No.: 16/336,724

(22) PCT Filed: Sep. 27, 2017
(86) PCT No.: PCT/US2017/053766

§ 371 Date: Mar. 26, 2019

Related U.S. Application Data
(60) Provisional application No. 62/552,296, filed on Aug. 30, 2017.
(60) Provisional application No. 62/459,340, filed on Feb. 15, 2017.
(60) Provisional application No. 62/457,952, filed on Feb. 12, 2017.
(60) Provisional application No. 62/400,481, filed on Sep. 27, 2016.

(51) Int. Cl. A61K 31/47; A61K 39/395; A61P 35/00

(52) U.S. Cl. CPC A61K 31/47; A61K 2039/545; A61P 35/00; A61K 39/3955

(57) ABSTRACT

The present disclosure relates to a method of treating urothelial carcinoma using Cabozantinib, a kinase inhibitor.

### RELATED APPLICATIONS

This application claims priority to U.S. Application Ser. No. 62/400,481, filed Sep. 27, 2016, U.S. Application Ser. No. 62/457,952, filed Feb. 12, 2017, U.S. Application Ser. No. 62/459,340, filed Feb. 15, 2017, and U.S. Application Ser. No. 62/552,296, filed Aug. 30, 2017. The entire contents of the aforementioned applications are incorporated herein by reference.

### TECHNICAL FIELD

The present disclosure relates to a method of treating cancer, including urothelial carcinoma, and other genitourinary malignancies in human patients using N-(4-(6,7-dimethoxyquinolin-4-yloxy)phenyl)-N′-(4-fluorophenyl)cyclopropane-1,1-dicarboxamide, a kinase inhibitor.

### BACKGROUND

Urothelial cancers encompass carcinomas of the bladder, ureters, and renal pelvis, which occur at a ratio of 50:3:1, respectively. According to the Cancer Network (cancernetwork.com/urothelial-and-kidney-cancers last visited Feb. 9, 2017), cancer of the urothelium is a multifocal process. Patients with cancer of the upper urinary tract have a 30% to 50% chance of developing cancer of the bladder at some point in their lives. On the other hand, patients with bladder cancer have a 2% to 3% chance of developing cancer of the upper urinary tract. The incidence of renal pelvis tumors is decreasing. In 2016, it was estimated that there will be 76,960 new diagnoses of bladder cancer in the United States, with approximately 16,390 deaths.

According to the Cancer Network, treatment of advanced metastatic urothelial cancer is generally considered to be palliative. Response rates have been shown to be high with cisplatin-containing regimens (50% to 60%), but the duration of response is short and median survival is 12 to 14 months. A randomized trial showed an advantage for a regimen of M-VAC (methotrexate, vinblastine, doxorubicin, and cisplatin) over cisplatin alone with regard to progression-free and overall survival, but with high rates of myelosuppression. In another randomized trial, the combination of gemcitabine and cisplatin exhibited survival equivalent to that with M-VAC in metastatic bladder cancer but was clinically better tolerated.

Because of the shortcomings of existing therapies, a need remains for new therapeutic options for the treatment of urothelial metastatic carcinoma and other genitourinary malignancies using new combinations of chemotherapeutic agents to improve response rates and minimize undesirable side effects.

### SUMMARY

These and other needs are met by the present invention, which is directed to a method of treating urothelial carcinoma and other genitourinary malignancies. The method employs Cabozantinib or a pharmaceutically acceptable salt thereof. Cabozantinib (S)-malate salt has been approved for the treatment of medullary thyroid cancer and advanced renal cell carcinoma. Cabozantinib has the structure depicted below.

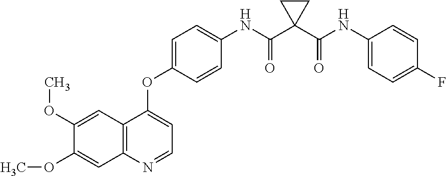 [SMILES: COc1cc2nccc(Oc3ccc(NC(=O)C4(C(=O)Nc5ccc(F)cc5)CC4)cc3)c2cc1OC]

Cabozantinib (S)-malate is commercially available in tablet form as CABOMETYX and in capsule form as COMETRIQ.

In one aspect, the invention is directed to a method of treating urothelial carcinoma in a human patient comprising administering to the human patient a therapeutically effective dose of Cabozantinib or a pharmaceutically acceptable salt thereof, alone or in combination with one or more therapeutic agents.

In another aspect, the invention is directed to a method of treating urothelial carcinoma in a human patient, comprising administering to the human patient a therapeutically effective dose of Cabozantinib or a pharmaceutically acceptable salt thereof alone or in combination with one or more therapeutic agent. The one or more agents can be selected from the group consisting of an anti-CTLA4 monoclonal antibody and an IgG4 anti-PD-1 monoclonal antibody.

In another aspect, the invention is directed to a method of treating a cancer selected from the group consisting of urothelial carcinoma, metastatic urothelial carcinoma, urachal adenocarcinoma, bladder squamous cell carcinoma of the bladder, castration-resistant prostate cancer, renal cell carcinoma-sarcomatoid tumor, trophoblastic tumor, germ cell cancer, and penile cancer, comprising administering to the human patient a therapeutically effective dose of Cabozantinib or a pharmaceutically acceptable salt thereof alone or in combination with one or more therapeutic agents. The one or more agents can be selected from the group consisting of the anti-CTLA4 monoclonal antibody ipilimumab and the IgG4 anti-PD-1 monoclonal antibody nivolumab.

In another aspect, the invention is directed to the use of cabozantinib or a pharmaceutically acceptable salt thereof for the treatment of a cancer selected from the group consisting of urothelial carcinoma, metastatic urothelial carcinoma, urachal adenocarcinoma, bladder squamous cell carcinoma of the bladder, castration-resistant prostate cancer, renal cell carcinoma-sarcomatoid tumor, trophoblastic tumor, germ cell cancer, and penile cancer.

In another aspect, the invention is directed to the use of a combination of cabozantinib or a pharmaceutically acceptable salt thereof and an anti-CTLA4 monoclonal antibody or an IgG4 anti-PD-1 monoclonal antibody or both an anti-CTLA4 monoclonal antibody and an IgG4 anti-PD-1 for the treatment of cancer. In one embodiment, the cancer is selected from group consisting of urothelial carcinoma, metastatic urothelial carcinoma, urachal adenocarcinoma, bladder squamous cell carcinoma of the bladder, castration-resistant prostate cancer, renal cell carcinoma-sarcomatoid tumor, trophoblastic tumor, germ cell cancer, and penile cancer. In one embodiment, the anti-CTLA4 monoclonal antibody is nivolumab and the IgG4 anti-PD-1 monoclonal antibody is ipilimumab.

In another aspect, the invention is directed to the use of a combination of cabozantinib or a pharmaceutically acceptable salt thereof and nivolumab for the treatment of cancer. In one embodiment, the cancer is selected from group consisting of urothelial carcinoma, metastatic urothelial carcinoma, urachal adenocarcinoma, bladder squamous cell carcinoma of the bladder, castration-resistant prostate cancer, renal cell carcinoma-sarcomatoid tumor, trophoblastic tumor, germ cell cancer, and penile cancer.

In another aspect, the invention is directed to the use of a combination of cabozantinib or a pharmaceutically acceptable salt thereof and nivolumab and ipilimumab for the treatment of cancer. In one embodiment, the cancer is selected from group consisting of urothelial carcinoma, metastatic urothelial carcinoma, urachal adenocarcinoma, bladder squamous cell carcinoma of the bladder, castration-resistant prostate cancer, renal cell carcinoma-sarcomatoid tumor, trophoblastic tumor, germ cell cancer, and penile cancer.

Ipilimumab is sold under the name YERVOY. It has previously been approved for the treatment of unresectable or metastatic melanoma. Ipilimumab is administered at a dose of 3 mg/kg administered intravenously over 90 minutes every 3 weeks for a total of 4 doses. (See packageinserts.bms.com/pi/piyervoy.pdf last visited Feb. 9, 2017).

Nivolumab is sold under the name OPDIVO. It has previously been approved for the treatment of unresectable or metastatic melanoma and disease progression following ipilimumab and, if BRAF V600 mutation positive, a BRAF inhibitor. Nivolumab is administered at a dose 3 mg/kg as an intravenous infusion over 60 minutes every 2 weeks. (2.1) (See www.accessdata.fda.gov/drugsatfda_docs/label/2014/1255541b1.pdf last visited Feb. 9, 2017)

In another embodiment, the invention is directed to a method of treating a cancer selected from the group consisting of bladder squamous cell carcinoma (bSCC) (squamous cell carcinoma of the bladder), urachal cancer, and penile cancer in a human patient comprising administering to the human patient a therapeutically effective dose of Cabozantinib alone or in combination with one or more therapeutic agent. The one or more agents can be selected from the group consisting of an anti-CTLA4 monoclonal antibody and an IgG4 anti-PD-1 monoclonal antibody.

In another embodiment, the invention is directed to a method of treating a cancer selected from the group consisting of bladder squamous cell carcinoma (bSCC) (squamous cell carcinoma of the bladder), squamous cell carcinoma, urachal adenocarcinoma, castration-resistant prostate cancer, renal cell carcinoma-sarcomatoid tumor, trophoblastic tumor, germ cell cancer, and penile cancer in a human patient, comprising administering to the human patient a therapeutically effective dose of Cabozantinib alone or in combination with one or more therapeutic agent. The one or more agents can be selected from the group consisting of the anti-CTLA4 monoclonal antibody ipilimumab and the IgG4 anti-PD-1 monoclonal antibody nivolumab.

These and other aspects and embodiments are described herein.

### BRIEF DESCRIPTION OF THE DRAWINGS

FIGS. 1A and 1B show that cabozantinib appears to decrease the Treg population in urothelial cancer patients.

FIGS. 2A and 2B demonstrate the biologic rationale for combining cabozantinib and immune checkpoint blockade.

FIG. 3 summarizes clinical treatment-adverse events reported during the study.

FIGS. 4 and 5 summarize laboratory abnormality events that were reported during the study.

FIGS. 6 and 7 summarize other treatment exposure events, dose discontinuation or dose adjustments during the study.

FIG. 8 depicts the tumor burden in patient target lesions for the combination of Cabozantinib+Nivolumab+/−Ipilimumab.

FIG. 9 shows the responses when the combination of Cabozantinib+Nivolumab (CaboNivo) and Cabozantinib+Nivolumab+Ipilimumab (CaboNivoIpi) was tested against various tumor types.

FIG. 10 shows the preliminary time to response and durability of response by cabozantinib dose (CR+PR).

FIG. 11 depicts median progression free survival.

FIG. 12 depicts median overall survival.

FIG. 13 depicts the study design for an expansion study of cabozantinib plus nivolumab (CaboNivo) alone or with Ipilimumab (CaboNivoIpi) in patients with metastatic urothelial carcinoma and other genitourinary tumors.

### DETAILED DESCRIPTION

Disclosed herein is a method of treating urothelial carcinoma, as well as other forms of cancer described herein, in a human patient, comprising administering to the human patient a therapeutically effective dose of Cabozantinib or a pharmaceutically acceptable salt thereof alone or in combination with one or more therapeutic agent. The one or more agents can be selected from the group consisting of an anti-CTLA4 monoclonal antibody and an IgG4 anti-PD-1 monoclonal antibody. The one or more agents can be selected from the group consisting of an anti-CTLA4 monoclonal antibody and an IgG4 anti-PD-1 monoclonal antibody. The one or more agents can be selected from the group consisting of the anti-CTLA4 monoclonal antibody ipilimumab and the IgG4 anti-PD-1 monoclonal antibody nivolumab. The form of cancer is selected from the group consisting of urothelial carcinoma, urachal adenocarcinoma, squamous cell carcinoma of the bladder, castration-resistant prostate cancer, renal cell carcinoma-sarcomatoid tumor, trophoblastic tumor, germ cell cancer, and penile cancer.

“Pharmaceutically acceptable salt” of a compound means a salt that is pharmaceutically acceptable and that possesses the desired pharmacological activity of the parent compound. It is understood that the pharmaceutically acceptable salts are non-toxic. Additional information on suitable pharmaceutically acceptable salts can be found in Remington's Pharmaceutical Sciences, 17^{th} ed., Mack Publishing Company, Easton, Pa., 1985, or in S. M. Berge, et al., “Pharmaceutical Salts,” J. Pharm. Sci., 1977; 66:1-19, both of which are incorporated herein by reference.

Examples of pharmaceutically acceptable acid addition salts include those formed with inorganic acids such as hydrochloric acid, hydrobromic acid, sulfuric acid, nitric acid, phosphoric acid, and the like; as well as organic acids such as acetic acid, trifluoroacetic acid, propionic acid, hexanoic acid, cyclopentanepropionic acid, glycolic acid, pyruvic acid, lactic acid, oxalic acid, maleic acid, malonic acid, succinic acid, fumaric acid, tartaric acid, malic acid, citric acid, benzoic acid, cinnamic acid, 3-(4-hydroxybenzoyl)benzoic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, p-toluenesulfonic acid, and salicylic acid and the like.

In one embodiment, cabozantinib is administered as a malate salt. In one embodiment, cabozantinib is administered as the (L)-malate salt (also referred to as the (S)-malate salt), or the (D)-malate salt (also referred to as the (R)-malate salt). The malate salt of cabozantinib is disclosed in PCT/US2010/021194 and U.S. Patent Application Ser. No. 61/325,095, the entire contents of each of which are incorporated herein by reference.

In another embodiment, the malate salt of cabozantinib is in the crystalline N-2 form of the (L)-malate salt and/or the (D)-malate salt of the as disclosed in U.S. Patent Application Ser. No. 61/325,095. See also WO 2008/083319 for the properties of crystalline enantiomers, including the N-1 and N-2 crystalline forms of the (L)-malate salt, (also referred to as the (S)-malate salt) or the (D)-malate salt (also referred to as the (R)-malate salt), and/or the N-1 crystalline forms of the (L)-malate salt (also referred to as the (S)-malate salt), or the (D)-malate salt (also referred to as the (R)-malate salt). Methods of making and characterizing such forms are fully described in PCT/US2010/021194, which is incorporated herein by reference in its entirety.

Cabozantinib or a pharmaceutically acceptable salt thereof can be administered as a pharmaceutical composition comprising at Cabozantinib or a pharmaceutically acceptable salt thereof and at least one pharmaceutically acceptable excipient. Pharmaceutical compositions of Cabozantinib have been disclosed in, for example, commonly assigned PCT Patent Publication Nos. WO 2005/030140, WO 2012/009722, and WO 2012/109510, each of which is incorporated by reference herein in its entirety.

The amount of the Cabozantinib or a pharmaceutically acceptable salt thereof in the pharmaceutical composition can be a therapeutically effective amount. The pharmaceutical composition can be a solid or dispersion pharmaceutical composition comprising at least one of a therapeutically effective amount Cabozantinib or a pharmaceutically acceptable salt thereof and a pharmaceutically acceptable excipient as described herein.

A pharmaceutical composition such as disclosed herein may be any pharmaceutical form which contains Cabozantinib or a pharmaceutically acceptable salt thereof. The pharmaceutical composition may be, for example, a tablet, capsule, liquid suspension, injectable, topical, or transdermal. The pharmaceutical compositions generally contain about 1% to about 99% by weight of the active compound(s), or a solid form of the active compound(s), and 99% to 1% by weight of a suitable pharmaceutical excipient. In one example, the composition will be between about 5% and about 75% by weight of active compound, with the rest being suitable pharmaceutical excipients or other adjuvants, as discussed below.

The actual amount required for treatment of any particular subject will depend upon a variety of factors including the disease state being treated and its severity; the specific pharmaceutical composition employed; the age, body weight, general health, sex, and diet of the subject; the mode of administration; the time of administration; the route of administration; and the rate of excretion of the active compound(s), or a solid form of the active compound(s), according to this disclosure; the duration of the treatment; any drugs used in combination or coincidental with the specific compound employed; and other such factors well known in the medical arts. These factors are discussed in Goodman and Gilman's “The Pharmacological Basis of Therapeutics,” Tenth Edition, A. Gilman, J. Hardman and L. Limbird, eds., McGraw-Hill Press, 155-173, 2001, which is incorporated herein by reference. The active compound(s), or a solid form of active compound(s), according to this disclosure and pharmaceutical compositions comprising them, may be used in combination with anticancer or other agents that are generally administered to a subject being treated for cancer. They may also be co-formulated with one or more of such agents in a single pharmaceutical composition.

Depending on the type of pharmaceutical composition, the pharmaceutically acceptable carrier may be chosen from any one or a combination of carriers known in the art. The choice of the pharmaceutically acceptable carrier depends partly upon the desired method of administration to be used. For a pharmaceutical composition of this disclosure, that is, one of the active compound(s), or a solid form of the active compound(s), of this disclosure, a carrier should be chosen so as to substantially maintain the particular form of the active compound(s), whether it would be solid or not. In other words, the carrier should not substantially alter the form of the active compound(s). Nor should the carrier be otherwise incompatible with the form of the active compound(s), such as by producing any undesirable biological effect or otherwise interacting in a deleterious manner with any other component(s) of the pharmaceutical composition.

Filler

As indicated above, the pharmaceutical composition containing Cabozantinib or a pharmaceutically acceptable salt thereof comprises a filler. Fillers are inert ingredients added to adjust the bulk in order to produce a size practical for compression. Examples of fillers include sodium starch glycolate, corn starch, talc, sucrose, dextrose, glucose, lactose, xylitol, fructose, sorbitol, calcium phosphate, calcium sulfate, calcium carbonate, and the like, or mixtures thereof. Microcrystalline cellulose may also be used as a filler and may be any suitable form of microcrystalline cellulose as is known and used in the tabletting art. Preferably, a mixture of lactose and microcrystalline cellulose is used as the filler. In one embodiment, the lactose is anhydrous lactose sold as Lactose 60M, which is readily commercially available from a number of suppliers. In one embodiment, the microcrystalline cellulose is Avicel PH-102, which is also commercially available.

Preferably, filler(s) are present in an amount of from about 50 to about 70 percent, and more preferably from about 57 to about 67 percent, by weight on a solids basis of the directly compressible formulation. Preferably, lactose is present in an amount of from about 18 to 22 percent by weight. Preferably, the microcrystalline cellulose is present in an amount of from about 38 to 40 percent by weight.

Binder

The pharmaceutical composition containing Cabozantinib or a pharmaceutically acceptable salt thereof also comprises a binder. Binders are added to powders to impart cohesive qualities to the powder, which allows the compressed tablet to retain its integrity. The binder can be any pharmaceutically acceptable binder available in the tabletting art, such as acacia, alginic acid, carbomer, carboxymethylcellulose sodium, dextrin, ethylcellulose, gelatin, guar gum, hydrogenated vegetable oil (type I), hydroxyethyl cellulose, hydroxypropyl cellulose, hydroxypropyl methylcellulose, liquid glucose, magnesium aluminium silicate, maltodextrin, methylcellulose, polymethacrylates, povidone, pregelatinized starch, sodium alginate, starch, zein, and the like, or mixtures thereof.

The preferred binder is hydroxypropyl cellulose preferably in an amount of from about 2 to about 4 percent by weight on a solid basis of the directly compressible formulation. In one embodiment, the hydroxypropyl cellulose is commercially available Klucel EXF.

Disintegrant

The pharmaceutical composition containing Cabozantinib or a pharmaceutically acceptable salt thereof also comprises a disintegrant. A disintegrant is a substance or a mixture of substances added to facilitate breakup or disintegrate after administration. The disintegrant may be any pharmaceutically acceptable disintegrant available in the tabletting art, including alginic acid, carboxymethylcellulose calcium, carboxymethylcellulose sodium, colloidal silicon dioxide, croscarmellose sodium, crospovidone, guar gum, magnesium aluminum silicate, methylcellulose, microcrystalline cellulose, polyacrilin potassium, powdered cellulose, pregelatinized starch, sodium alginate, starch, and the like, or mixtures thereof.

The preferred disintegrant is croscarmellose sodium, in an amount of from about 4 to about 8 percent by weight, on a solid basis of the directly compressible formulation. In one embodiment, the croscarmellose sodium is commercially available Ac-Di-Sol.

Glidant

The pharmaceutical composition containing Cabozantinib or a pharmaceutically acceptable salt thereof also comprises a glidant. The glidant may be any pharmaceutically acceptable glidant which contributes to the compressibility, flowability, and homogeneity of the formulation and which minimizes segregation and does not significantly interfere with the release mechanism of the binders as set forth above. Preferably, the glidant is selected to improve the flow of the formulation. Silicon dioxide, particularly colloidal silicon dioxide, is preferred as a glidant.

The glidant is used in an amount of from about 0.2 to about 0.6 percent by weight on a solid basis of the directly compressible formulation.

Lubricant

The pharmaceutical composition containing Cabozantinib or a pharmaceutically acceptable salt thereof also comprises a lubricant. Lubricants are employed to prevent adhesion of the tablet material to the surface of dyes and punches. The lubricant may be any pharmaceutically acceptable lubricant which substantially prevents segregation of the powder by contributing to homogeneity of the formulation and which exhibits good flowability. Preferably, the lubricant functions to facilitate compression of the tablets and ejection of the tablets from the die cavity. Such lubricants may be hydrophilic or hydrophobic, and examples include magnesium stearate, Lubritab®, stearic acid, talc, and other lubricants known in the art or to be developed which exhibit acceptable or comparable properties, or mixtures thereof. Examples of lubricants include calcium stearate, glyceryl monostearate, glyceryl palmitostearate, hydrogenated castor oil, hydrogenated vegetable oil, light mineral oil, magnesium stearate, mineral oil, polyethylene glycol, sodium benzoate, sodium lauryl sulfate, sodium stearyl fumarate, stearic acid, talc, zinc stearate, and the like, or mixtures thereof.

The lubricant should be selected to aid in the flow of the powder in the hopper and into the die. Magnesium stearate exhibits excellent properties in combination with the other preferred excipients of the formulation. Magnesium stearate contributes to reducing friction between the die wall and tablet formulation during compression, as well as to the easy ejection of the Cabozantinib tablets. It also resists adhesion to punches and dies.

Preferably, the lubricant is magnesium stearate (non-bovine) used in an amount of from about 0.5 to about 1.0 percent by weight on a solid basis of the directly compressible formulation.

Film Coating

The pharmaceutical composition containing Cabozantinib or a pharmaceutically acceptable salt thereof can also comprise an optional film coating when it is a tablet. The film coat concentration can be about 1 to about 10 percent by weight on a solid basis of the directly compressible formulation. Film coating suspensions may include combinations of the following components: hypromeollose, carboxymethylcellulose sodium, carnauba wax, cellulose acetate phthalate, cetyl alcohol, confectioner's sugar, ethyl cellulose, gelatin, hydroxyethyl cellulose, hydroxypropyl cellulose, hydroxypropyl methylcellulose, liquid glucose, maltodextrin, methyl cellulose, microcrystalline wax, Opadry and Opadry II, polymethacrylates, polyvinyl alcohol, shellac, sucrose, talc, titanium dioxide, and zein.

Other Adjuvants

Other pharmaceutically acceptable adjuvants known in the pharmaceutical formulation art may also be used in the pharmaceutical compositions of this disclosure. These include, but are not limited to, preserving, wetting, suspending, sweetening, flavoring, perfuming, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. If desired, a pharmaceutical composition of this disclosure may also contain minor amounts of auxiliary substances such as wetting or emulsifying agents, pH buffering agents, and antioxidants, such as, for example, citric acid, sorbitan monolaurate, triethanolamine oleate, and butylated hydroxytoluene

The pharmaceutical compositions of this disclosure may be prepared by methods known in the pharmaceutical formulation art, for example, see Remington's Pharmaceutical Sciences, 18^{th} Ed., (Mack Publishing Company, Easton, Pa., 1990). In solid dosage forms, any one of Forms 1-27, or combinations thereof, is admixed with at least one pharmaceutically acceptable excipient such as sodium citrate or dicalcium phosphate or (a) fillers or extenders, as for example, starches, lactose, sucrose, glucose, mannitol, and silicic acid, (b) binders, as for example, cellulose derivatives, starch, alginates, gelatin, polyvinylpyrrolidone, sucrose, and gum acacia, (c) humectants, as for example, glycerol, (d) disintegrating agents, as for example, agar-agar, calcium carbonate, potato or tapioca starch, alginic acid, croscarmellose sodium, complex silicates, and sodium carbonate, I solution retarders, as for example paraffin, (f) absorption accelerators, as for example, quaternary ammonium compounds, (g) wetting agents, as for example, cetyl alcohol, and glycerol monostearate, magnesium stearate, and the like (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

In some instances, the pharmaceutical dosage form may be a solid dispersion. The term “solid dispersion” refers to a system in a solid state comprising at least two components, wherein one component is dispersed throughout the other component or components. In some instances, the pharmaceutical dosage form may be an amorphous solid dispersion. The term “amorphous solid dispersion”, as used herein, refers to stable solid dispersions comprising amorphous drug substance (Cabozantinib) and a stabilizing polymer. By “amorphous drug substance,” it is meant that the amorphous solid dispersion contains a drug substance in a substantially amorphous solid form—that is at least 80% of the drug substance in the dispersion is in an amorphous form. More preferably, at least 90% and most preferably at least 95% of the drug substance in the dispersion is in amorphous form. The term “stabilizing polymer”, as used herein, refers to any polymer known to the skilled practitioner that is used to stabilize an amorphous drug substance in a solid dispersion such as are described, for instance, in Remington's Pharmaceutical Sciences, 18^{th} Ed., (Mack Publishing Company, Easton, Pa., 1990).

Processes for making such solid dispersions are also available to the skilled practitioner and include, for instance, spray drying, melt extrusion, freeze drying, rotary evaporation, drum drying, or other solvent removal processes. In the spray drying process, the amorphous dispersion is formed by dispersing or dissolving the drug substance and the stabilizing polymer in a suitable solvent to form a feed solution, pumping the feed solution through an atomizer into a drying chamber, and removing the solvent to form the amorphous solid dispersion powder in the drying chamber. A drying chamber uses hot gases, such as forced air, nitrogen, nitrogen-enriched air, or argon to dry particles. The feed solution can be atomized by conventional means well known in the art, such as a two-fluid sonicating nozzle and a two-fluid non-sonicating nozzle.

Solid dosage forms as described above can be prepared with coatings and shells, such as enteric coatings and others well known in the art. They may contain pacifying agents, and can also be of such composition that they release the active compound or compounds in a certain part of the intestinal tract in a delayed manner. Examples of embedded compositions that can be used are polymeric substances and waxes. The active compounds can also be in microencapsulated form, if appropriate, with one or more of the above-mentioned excipients.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are, for example, suppositories that can be prepared by mixing the active compound(s), or a solid form of the active compound(s), with, for example, suitable non-irritating excipients or carriers such as cocoa butter, polyethyleneglycol, or a suppository wax, which are solid at ordinary temperatures but liquid at body temperature and therefore melt while in a suitable body cavity and release the active component therein.

Solid dosage forms are preferred for the pharmaceutical composition of this disclosure. Solid dosage forms for oral administration, which includes capsules, tablets, pills, powders, and granules, are particularly preferred. In such solid dosage forms, the active compound(s) mixed with at least one inert, pharmaceutically acceptable excipient (also known as a pharmaceutically acceptable carrier). Administration of the active compound(s), or a solid form of the active compound(s), in pure form or in an appropriate pharmaceutical composition, can be carried out via any of the accepted modes of administration or agents for serving similar utilities. Thus, administration can be, for example, orally, nasally, parenterally (intravenous, intramuscular, or subcutaneous), topically, transdermally, intravaginally, intravesically, intracistemally, or rectally, in the form of solid, semi-solid, lyophilized powder, or liquid dosage forms, such as for example, tablets, suppositories, pills, soft elastic and hard gelatin capsules, powders, solutions, suspensions, or aerosols, or the like, preferably in unit dosage forms suitable for simple administration of precise dosages. One preferable route of administration is oral administration, using a convenient dosage regimen that can be adjusted according to the degree of severity of the disease-state to be treated. For example, the dosage regimen can be as a capsule or tablet for oral administration.

The skilled artisan will recognize that a greater amount of Cabozantinib as a salt as described herein is present to provide a certain amount of Cabozantinib. For example, the molecular weight of Cabozantinib is 501.51 and the molecular weight of Cabozantinib (S)-malate salt is 635.60. Thus, 100 mg of (S)-malate salt is required to provide 80 mg of Cabozantinib. The “free base equivalent” (the) of a tablet or capsule containing 100 mg of Cabozantinib, (S)-malate is 80 mg Cabozantinib. Proportionally smaller or larger amounts of Cabozantinib as a salt are required for tablet or capsule compositions containing less or more of Cabozantinib.

The pharmaceutical composition contemplated for use comprises Cabozantinib or a pharmaceutically acceptable salt thereof and a pharmaceutically acceptable carrier. The compositions contain less than 100 ppm of 6,7-dimethoxy-quinoline-4-ol, the structure of which is

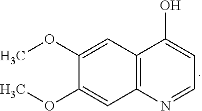

Minimizing the concentration of degradation products, contaminants, or byproducts such as 6,7-dimethoxy-quinoline-4-ol in pharmaceutical compositions destined for human administration is desirable. In one embodiment, a pharmaceutical composition comprises Cabozantinib or a pharmaceutically acceptable salt thereof as disclosed herein and a pharmaceutically acceptable carrier containing less than 90 ppm, less than 80 ppm, less than 70 ppm, less than 60 ppm, less than 50 ppm, less than 40 ppm, less than 30 ppm, less than 20 ppm, less than 10 ppm, less than 5 ppm, less than 2.5 ppm of 6,7-dimethoxy-quinoline-4-ol, the structure of which is

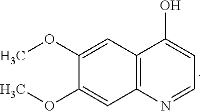

In another, a pharmaceutical composition comprises Cabozantinib or a pharmaceutically acceptable salt thereof and a pharmaceutically acceptable carrier containing 1 to 100 ppm, 1 to 80 ppm, 1 to 60 ppm, 1 to 40 ppm, 1 to 20 ppm, 1 to 10 ppm, 1 to 5 ppm, 1 to 2.5 ppm of 6,7-dimethoxy-quinoline-4-ol, the structure of which is

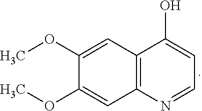

In another, a pharmaceutical composition comprises Cabozantinib or a pharmaceutically acceptable salt thereof and a pharmaceutically acceptable carrier containing 0.1 to 100 ppm, 0.1 to 80 ppm, 0.1 to 60 ppm, 0.1 to 40 ppm, 0.1 to 20 ppm, 0.1 to 10 ppm, 0.1 to 5 ppm, 0.1 to 2.5 ppm, or 0.1 to 1 ppm of 6,7-dimethoxy-quinoline-4-ol, the structure of which is

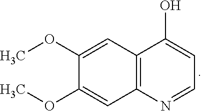

Capsule Formulation

In one embodiment, the dosage regimen is as a capsule formulation for oral administration.

In one embodiment, the capsule formulation comprises:
5-60 percent by weight of Cabozantinib or a pharmaceutically acceptable salt thereof;
30-80 percent by weight of one or more fillers;
1-15 percent by weight of one or more disintegrants;
0.1 to 1.0 percent by weight of a glidant; and
0.1 to 4.0 percent by weight of a lubricant.

In another embodiment, the capsule formulation comprises:
5-60 percent by weight of Cabozantinib or a pharmaceutically acceptable salt thereof;
30-80 percent by weight of one or more fillers;
2-12 percent by weight of one or more disintegrants;
0.1 to 0.6 percent by weight of a glidant; and
0.1 to 3.0 percent by weight of a lubricant.

In another embodiment, the capsule formulation comprises:
5-15 percent by weight of Cabozantinib or a pharmaceutically acceptable salt thereof;
70-80 percent by weight of one or more fillers;
8-12 percent by weight of one or more disintegrants;
0.1 to 0.4 percent by weight of a glidant; and
0.1 to 2.0 percent by weight of a lubricant.

In another embodiment, the capsule formulation comprises:
5-15 percent by weight of Cabozantinib or a pharmaceutically acceptable salt thereof;
70-80 percent by weight of one or more fillers;
9-11 percent by weight of one or more disintegrants;
0.2 to 0.4 percent by weight of a glidant; and
0.5 to 1.5 percent by weight of a lubricant.

In another embodiment, the capsule formulation comprises:
40-60 percent by weight of Cabozantinib or a pharmaceutically acceptable salt thereof;
30-50 percent by weight of one or more fillers;
2-12 percent by weight of one or more disintegrants;
0.1 to 0.6 percent by weight of a glidant; and
0.1 to 3.0 percent by weight of a lubricant.

In another embodiment, the capsule formulation comprises:
45-55 percent by weight of Cabozantinib or a pharmaceutically acceptable salt thereof;
35-40 percent by weight of one or more fillers;
8-12 percent by weight of one or more disintegrants;
0.2 to 0.5 percent by weight of a glidant; and
0.5 to 2.5 percent by weight of a lubricant.

In one embodiment, the capsule formulation comprises:
5-60 percent by weight of Cabozantinib or a pharmaceutically acceptable salt thereof;
30-80 percent by weight of microcrystalline cellulose;
2-7 percent by weight of croscarmellose sodium;
2-7 percent by weight of sodium starch glycolate;
0.1 to 1.0 percent by weight of a fumed silica; and
0.1 to 4.0 percent by weight of stearic acid.

In another embodiment, the capsule formulation comprises:
5-60 percent by weight of Cabozantinib or a pharmaceutically acceptable salt thereof;
30-80 percent by weight of microcrystalline cellulose;
3-6 percent by weight of croscarmellose sodium;
3-6 percent by weight of sodium starch glycolate;
0.1 to 0.6 percent by weight of fumed silica; and
0.1 to 3.0 percent by weight of stearic acid.

In another embodiment, the capsule formulation comprises:
5-15 percent by weight of Cabozantinib or a pharmaceutically acceptable salt thereof;
70-80 percent by weight of microcrystalline cellulose;
4-6 percent by weight of croscarmellose sodium;
4-6 percent by weight of sodium starch glycolate;
0.1 to 0.4 percent by weight of fumed silica; and
0.1 to 2.0 percent by weight of stearic acid.

In another embodiment, the capsule formulation comprises:
5-15 percent by weight of Cabozantinib or a pharmaceutically acceptable salt thereof;
70-80 percent by weight of microcrystalline cellulose;
4.5-5.5 percent by weight of croscarmellose sodium;
4.5-5.5 percent by weight of sodium starch glycolate;
0.2 to 0.4 percent by weight of fumed silica; and
0.5 to 1.5 percent by weight of stearic acid.

In another embodiment, the capsule formulation comprises:
40-60 percent by weight of Cabozantinib or a pharmaceutically acceptable salt thereof;
30-50 percent by weight of microcrystalline cellulose;
2-7 percent by weight of croscarmellose sodium;
2-7 percent by weight of sodium starch glycolate;
0.1 to 0.6 percent by weight of fumed silica; and
0.1 to 3.0 percent by weight of stearic acid.

In another embodiment, the capsule formulation comprises:
45-55 percent by weight of Cabozantinib or a pharmaceutically acceptable salt thereof;
35-40 percent by weight of microcrystalline cellulose;
3-6 percent by weight of croscarmellose sodium;
3-6 percent by weight of sodium starch glycolate;
0.2 to 0.5 percent by weight of fumed silica; and
0.5 to 2.5 percent by weight of stearic acid.

In one embodiment, the capsule compositions of this disclosure contain from 5 to about 200 mg of Cabozantinib in at least one of the forms described herein. In another embodiment, the capsule compositions of this disclosure contain 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 mg of Cabozantinib. In another embodiment, the capsule compositions of this disclosure contain from 105 to 200 mg of Cabozantinib. In another embodiment, the capsule compositions of this disclosure contain 105, 110, 115, 120, 125, 130, 135, 140, 145, 150, 155, 160, 165, 170, 175, 180, 185, 190, 195, or 200 mg of Cabozantinib. In another embodiment, the capsule compositions of this disclosure contain from 20 to 100 mg of Cabozantinib. In another embodiment, the capsule compositions of this disclosure contain 5, 10, 15, 20, 25, 30, 35, 40, 45, 50, 55, 60, 65, 70, 75, 80, 85, 90, 95, or 100 mg of Cabozantinib. In another embodiment, the capsule compositions of this disclosure contain from 20 to 60 mg of Cabozantinib. In another embodiment, the capsule compositions of this disclosure contain 5, 10, 15, 20, 25, 30, 35, 40, 45, 50, 55, 60 mg Cabozantinib. In another embodiment, the capsule compositions contain 20, 25, 40, 50, 60, 75, 80, or 100 mg of Cabozantinib. In another embodiment, the capsule compositions of this disclosure contain 20 mg of Cabozantinib. In another embodiment, the capsule compositions of this disclosure contain 40 mg of Cabozantinib. In another embodiment, the capsule compositions of this disclosure contain 60 mg of Cabozantinib. In another embodiment, the capsule compositions of this disclosure contain 80 mg of Cabozantinib.

In another aspect, the disclosure provides a pharmaceutical capsule composition according to Table 1.

TABLE 1

| Ingredient | mg/unit dose |
|---|---|
| Cabozantinib or a pharmaceutically acceptable | 25 |
| salt thereof (10% drug load as Cabozantinib) | |
| Silicified Microcrystalline Cellulose | 196.75 |
| Croscarmellose sodium | 12.5 |
| Sodium starch glycolate | 12.5 |
| Fumed Silica | 0.75 |
| Stearic acid | 2.5 |
| Total Fill Weight | 250 |

In another aspect, the disclosure provides a pharmaceutical capsule composition according to Table 2.

TABLE 2

| Ingredient | mg/unit dose |
|---|---|
| Cabozantinib or a pharmaceutically acceptable | 100 |
| salt thereof (50% drug load as Cabozantinib) | |
| Silicified Microcrystalline Cellulose | 75.40 |
| Croscarmellose sodium | 10.00 |
| Sodium Starch Glycolate | 10.00 |
| Fumed silica | 0.6 |
| Stearic Acid | 4.0 |
| Total Fill Weight | 200 |

The capsule formulations can be prepared according to methods available to the skilled person, by combining and mixing the components of the formulation and filling two-piece hard gelatin capsules. The capsule shell ingredients include gelatin and optionally colorant.

Tablet Formulation

In one embodiment, the dosage regimen is as a tablet formulation for oral administration.

In one embodiment, the tablet formulation comprises:
25-40 percent by weight of Cabozantinib or a pharmaceutically acceptable salt thereof;
45-75 percent by weight of one or more diluents;
1-5 percent by weight of a binder;
2-10 percent by weight a disintegrant;
0.05-1.0 percent by weight of a glidant; and
0.5-1 percent by weight of a lubricant.

In another embodiment, the tablet composition comprises
28-38 percent by weight of Cabozantinib or a pharmaceutically acceptable salt thereof;
48-68 percent by weight of one or more diluents;
1.5-4.5 percent by weight of a binder;
3-9 percent by weight a disintegrant;
0.1-0.8 percent by weight of a glidant; and
0.5-1 percent by weight of a lubricant.

In another embodiment, the tablet composition comprises:
30-32 percent by weight of Cabozantinib or a pharmaceutically acceptable salt thereof;
50-70 percent by weight of one or more diluents;
2-4 percent by weight of a binder;
4-8 percent by weight a disintegrant;
0.2-0.6 percent by weigh of a glidant; and
0.5-1 percent by weight of a lubricant; wherein the composition is coated.

In one embodiment, the tablet formulation comprises:
25-40 percent by weight of Cabozantinib or a pharmaceutically acceptable salt thereof;
35-45 percent by weight or microcrystalline cellulose;
15 to 25 percent by weight of lactose anhydrous;
1-5 percent by weight of hydroxypropyl cellulose;
2-10 percent by weight croscarmellose sodium;
0.05-1.0 percent by weight of a colloidal silicon dioxide; and
0.5-1 percent by weight magnesium stearate.

In another embodiment, the tablet composition comprises
28-38 percent by weight of Cabozantinib or a pharmaceutically acceptable salt thereof;
36-42 percent by weight or microcrystalline cellulose;
18 to 22 percent by weight of lactose anhydrous;
1.5-4.5 percent by weight of a hydroxypropyl cellulose;
3-9 percent by weight a croscarmellose sodium;
0.1-0.8 percent by weight of a colloidal silicon dioxide; and
0.5-1 percent by weight of a magnesium stearate.

In another embodiment, the tablet composition comprises
28-38 percent by weight of Cabozantinib or a pharmaceutically acceptable salt thereof;
37-39 percent by weight or microcrystalline cellulose;
18 to 20 percent by weight of lactose anhydrous;
1.5-4.5 percent by weight of hydroxypropyl cellulose;
3-9 percent by weight a croscarmellose sodium;
0.1-0.8 percent by weight of colloidal silicon dioxide; and
0.5-1 percent by weight of magnesium stearate.

In another embodiment, the tablet composition comprises:
30-32 percent by weight of Cabozantinib or a pharmaceutically acceptable salt thereof;
38-39 percent by weight or microcrystalline cellulose;
19 to 20 percent by weight of lactose anhydrous;
2-4 percent by weight of hydroxypropyl cellulose;
4-8 percent by weight a croscarmellose sodium;
0.2-0.6 percent by weigh of colloidal silicon dioxide; and
0.5-1 percent by weight of a magnesium stearate.

The tablet formulations of these and other embodiments can be coated. Many coatings are known to the skilled person. An example of a coating is OPADRY Yellow, which contains hypromellose, titanium dioxide, triacetin, and iron oxide yellow.

In one embodiment, the tablet compositions of this disclosure contain from 5 to about 200 mg of Cabozantinib in at least one of the forms described herein. In another embodiment, the tablet compositions of this disclosure contain 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 mg of Cabozantinib. In another embodiment, the tablet compositions of this disclosure contain from 105 to 200 mg of Cabozantinib. In another embodiment, the tablet compositions of this disclosure contain 105, 110, 115, 120, 125, 130, 135, 140, 145, 150, 155, 160, 165, 170, 175, 180, 185, 190, 195, or 200 mg of Cabozantinib. In another embodiment, the tablet compositions of this disclosure contain from 20 to 100 mg of Cabozantinib. In another embodiment, the tablet compositions of this disclosure contain 5, 10, 15, 20, 25, 30, 35, 40, 45, 50, 55, 60, 65, 70, 75, 80, 85, 90, 95, or 100 mg of Cabozantinib. In another embodiment, the tablet compositions of this disclosure contain from 20 to 60 mg of Cabozantinib. In another embodiment, the tablet compositions of this disclosure contain 5, 10, 15, 20, 25, 30, 35, 40, 45, 50, 55, 60 mg Cabozantinib. In another embodiment, the tablet compositions contain 20, 25, 40, 50, 60, 75, 80, or 100 mg of Cabozantinib. In another embodiment, the tablet compositions of this disclosure contain 20 mg of Cabozantinib. In another embodiment, the tablet compositions of this disclosure contain 40 mg of Cabozantinib. In another embodiment, the tablet compositions of this disclosure contain 60 mg of Cabozantinib.

In another embodiment, the once-daily tablet comprises:

| Ingredient | (% w/w) |
|---|---|
| Cabozantinib or a pharmaceutically | 31.68 |
| acceptable salt thereof (based on free | |
| base) | |
| Microcrystalline Cellulose | 38.85 |
| Lactose anhydrous | 19.42 |
| Hydroxypropyl Cellulose | 3.00 |
| Croscarmellose Sodium | 3.00 |
| Total Intra-granular | 95.95 |
| Silicon dioxide, Colloidal | 0.30 |
| Croscarmellose Sodium | 3.00 |
| Magnesium Stearate | 0.75 |
| Total | 100.00 |

In another embodiment, the once-daily tablet formulation comprises:

| Ingredient | (% w/w) |
|---|---|
| Cabozantinib or a pharmaceutically | 25.0-33.3 |
| acceptable salt thereof (based on free | |
| base) | |
| Microcrystalline Cellulose | q.s. |
| Hydroxypropyl Cellulose | 3 |
| Poloxamer | 0-3 |
| Croscarmellose Sodium | 6.0 |
| Colloidal Silicon Dioxide | 0.5 |
| Magnesium Stearate | 0.5-1.0 |
| Total | 100 |

In another embodiment, the once-daily tablet or capsule formulation comprises:

| | Theoretical |
|---|---|
| | Quantity |
| Ingredient | (mg/unit dose) |
| Cabozantinib or a pharmaceutically | 100.0 |
| acceptable salt thereof (based on free | |
| base) | |
| Microcrystalline Cellulose PH-102 | 155.4 |
| Lactose Anhydrous 60M | 77.7 |
| Hydroxypropyl Cellulose, EXF | 12.0 |
| Croscarmellose Sodium | 24 |
| Colloidal Silicon Dioxide | 1.2 |
| Magnesium Stearate (Non-Bovine) | 3.0 |
| Opadry Yellow | 16.0 |
| Total | 416 |

In another embodiment, the once-daily tablet or capsule formulation comprises:

| Ingredient | % w/w |
|---|---|
| Cabozantinib or a pharmaceutically | 31.7 |
| acceptable salt thereof (based on free | |
| base) | |
| Microcrystalline Cellulose (Avicel PH- | 38.9 |
| 102) | |
| Lactose Anhydrous (60M) | 19.4 |
| Hydroxypropyl Cellulose (EXF) | 3.0 |
| Croscarmellose Sodium (Ac-Di-Sol) | 6.0 |
| Colloidal Silicon Dioxide | 0.3 |
| Magnesium Stearate | 0.75 |
| Opadry Yellow Film Coating which | 4.00 |
| includes: | |
| HPMC 2910/Hypromellose 6 cp | |
| Titanium dioxide | |
| Triacetin | |
| Iron Oxide Yellow | |

Processes for making the tablet formulation comprises mixing Cabozantinib or a pharmaceutically acceptable salt thereof with one or more of the pharmaceutical excipients. The mixture is then taken up in an aqueous solution containing a binder to form a binder solution. The binder solution is granulated using a granulation technique known in the art. For example, the granulation method may comprise wet high shear granulation using a wet high shear granulator. The resulting wet granules are then screened and dried using fluid bed drying or the like. The dried granules are then milled. The resulting dry milled granules are then mixed with a glidant and a disintegrant to form an extra-granular blend. A lubricant is then blended into the extraganular blend to form the final blend. The final blend is subsequently compressed to form the compressed tablet, which may be film coated.

More particularly, the process for making the tablet formulation comprises delumping Cabozantinib or a pharmaceutically acceptable salt thereof as needed prior to mixing with the excipients. Delumping ensures that the Cabozantinib or a pharmaceutically acceptable salt thereof mixes homogeneously with the other excipients during the formulation process. Delumped Cabozantinib or a pharmaceutically acceptable salt thereof is then mixed with microcrystalline cellulose, such as Avicel PH102, lactose (anhydrous, 60M), and croscarmellose sodium. This mixture is then combined with EXF grade hydroxypropoyl cellulose in water to form a binder solution, which is then wet high shear granulated. The resulting wet granules are wet screened and then fluid bed dried according to methods available to the skilled artisan. The resulting dried granules are milled and combined with colloidal silicon dioxide and croscarmellose sodium. Magnesium stearate is added to the mixture. This final blend is then ready for tablet compression. The resulting uncoated core tablets are subsequently film coated. The film coating comprises Opadry Yellow, which contains hypromellose, titanium dioxide, triacetin, and iron oxide yellow.

More particularly, the formulation process comprises:
a) Delumping unmilled Cabozantinib or a pharmaceutically acceptable salt thereof;
b) Premixing the delumped Cabozantinib or a pharmaceutically acceptable salt thereof or a pharmaceutically acceptable salt thereof with Avicel PH102, lactose anhydrous 60M, and croscarmellose sodium to form a binder solution;
c) Wet high shear granulation of the binder solution to produce wet granules;
d) Wet screening of the wet granules to produce wet screened granules;
e) Fluid bed drying of the wet screened granules to produce dried granules;
f) Dry milling of the dried granules to produce dried milled granules;
g) Blending the dried milled granules with colloidal silicon and croscarmellose to produce an extragranular blend;
h) Lubricant blending of the extragranular blend and magnesium stearate to produce a final blend;
i) Tablet compression of the final blend to form an uncoated core tablet; and
j) Film coating of the uncoated core tablet.

Cabozantininb can be administered as a CABOMETYX tablet. CABOMETYX is a tablet comprising Cabozantinib (S)-malate, microcrystalline cellulose, anhydrous lactose, hydroxypropyl cellulose, croscarmellose sodium, colloidal silicon dioxide magnesium stearate, and film coating comprising hypromellose, titanium dioxide, triacetin, and iron oxide yellow.

In an embodiment, the Cabozantinib (S)-malate is administered as a tablet formulation comprising approximately:
30-32 percent by weight of Cabozantinib, (S)-malate salt;
38-40 percent by weight of microcrystalline cellulose;
18-22 percent by weight of lactose;
2-4 percent by weight of hydroxypropyl cellulose;
4-8 percent by weight of croscarmellose sodium;
0.2-0.6 percent by weight of colloidal silicon dioxide;
0.5-1 percent by weight of magnesium stearate; and further comprising:

a film coating material comprising hypromellose, titanium dioxide, triacetin, and iron oxide yellow.

In another embodiment, the Cabozantinib (S)-malate is administered as a tablet formulation comprises approximately:
31-32 percent by weight of Cabozantinib, (S)-malate salt;
39-40 percent by weight of microcrystalline cellulose;
19-20 percent by weight of lactose;
2.5-3.5 percent by weight of hydroxypropyl cellulose;
5.5-6.5 percent by weight of croscarmellose sodium;
0.25-0.35 percent by weight of colloidal silicon dioxide;
0.7-0.8 percent by weight of magnesium stearate; and further comprising:
3.9-4.1 percent by weight of a film coating material comprising hypromellose, titanium dioxide, triacetin, and iron oxide yellow.

In another embodiment the Cabozantinib is administered as the (S)-malate as a tablet formulation selected from the group consisting of:

| | Theoretical Quantity (mg/unit dose) | | |
|---|---|---|---|
| | 20-mg | 40-mg | 60-mg |
| Ingredient | Tablet | Tablet | Tablet |
| Cabozantinib (S)-malate | 25.34 | 50.69 | 76.03 |
| Microcrystalline Cellulose, PH-102 | 31.08 | 62.16 | 93.24 |
| Lactose Anhydrous, 60M | 15.54 | 31.07 | 46.61 |
| Hydroxypropyl Cellulose, EXF | 2.400 | 4.800 | 7.200 |
| Croscarmellose Sodium | 4.800 | 9.600 | 14.40 |
| Colloidal Silicon Dioxide | 0.2400 | 0.4800 | 0.7200 |
| Magnesium Stearate (Non-Bovine) | 0.6000 | 1.200 | 1.800 |
| Opadry ® Yellow (03K92254) | 3.200 | 6.400 | 9.600 |
| Total tablet weight | 83.20 | 166.4 | 249.6 |

In another embodiment, the Cabozantinib (S)-malate is administered to treat urothelial carcinoma disclosed herein as a tablet formulation containing 20, 40, or 60 mg of Cabozantinib. 60 mg tablets are yellow film-coated, oval shaped with no score, debossed with “XL” on one side and “60” on the other side of the tablet; available in bottles of 30 tablets: NDC 42388-023-26. 40 mg tablets are yellow film-coated, triangle shaped with no score, debossed with “XL” on one side and “40” on the other side of the tablet; available in bottles of 30 tablets: NDC 42388-025-26. 20 mg tablets are yellow film-coated, round shaped with no score, debossed with “XL” on one side and “20” on the other side of the tablet; available in bottles of 30 tablets: NDC 42388-024-26.

In a further embodiment, the 60 mg of Cabozantinib is administered once daily as the CABOMETYX tablet formulation as described herein.

In another embodiment, the invention relates to the treatment of urothelial carcinoma, comprising administering to a patient in need of such treatment a therapeutically effective amount of Cabozantinib or a pharmaceutically acceptable salt thereof. In one embodiment, the urothelial carcinoma is metastatic urothelial carcinoma. In one embodiment, the metastatic urothelial carcinoma is relapsed or refractory. In one embodiment, the Cabozantinib is administered at a dose of 20-60 mg once daily. In another embodiment, Cabozantinib or a pharmaceutically acceptable salt thereof is administered at a dose of 20 mg once daily. In another embodiment, Cabozantinib or a pharmaceutically acceptable salt thereof is administered at a dose of 40 mg once daily. In another embodiment, Cabozantinib or a pharmaceutically acceptable salt thereof is administered at a dose of 60 mg once daily. In another embodiment, the Cabozantinib is administered as the (S)-malate. In another embodiment, the Cabozantinib is administered as Cabometyx tablets. In one embodiment, ORR was improved in patients with lung and/or liver metastases as compared to patients without lung and/or liver metastases.

In another embodiment, the invention relates to the treatment of urothelial carcinoma, comprising administering to a patient in need of such treatment, a therapeutically effective amount of Cabozantinib or a pharmaceutically acceptable salt thereof. In one embodiment, the urothelial carcinoma is relapsed or refractory. In one embodiment, the Cabozantinib or a pharmaceutically acceptable salt thereof is administered at a dose of 20-60 mg once daily. In another embodiment, Cabozantinib or a pharmaceutically acceptable salt thereof is administered at a dose of 20 mg once daily. In another embodiment, Cabozantinib or a pharmaceutically acceptable salt thereof is administered at a dose of 40 mg once daily. In another embodiment, Cabozantinib or a pharmaceutically acceptable salt thereof is administered at a dose of 60 mg once daily. In another embodiment, the Cabozantinib is administered as the (S)-malate. In another embodiment, the Cabozantinib is administered as Cabometyx tablets.

In these and other embodiments, the urothelial carcinoma can be urothelial carcinoma metastasized to other tissue. The other tissue can be, for example, bone tissue or brain tissue, although all metastases are contemplated. When the urothelial carcinoma is metastasized to bone, bone turnover markers can be used to evaluate the effect of Cabozantinib on urothelial carcinoma and bone metastases. The bone turnover markers that can be used include BTM [serum C-telopeptide (CTx), osteocalcin, N-telopeptide (NTx), procollagen type 1 (Pc 1) and urinary NTx] as assessed by NaF PET or Tc-MDP assay. In these and other embodiments, treatment with cabozantinib as described herein leads to a decline in concentration of one or more of these bone turnover markers.

In these and other embodiments, the Cabozantinib as a pharmaceutical formulation as described herein can be administered post sorafenib or lenvatinib therapy. In another embodiment, the Cabozantinib as a pharmaceutical formulation as described herein can be administered in combination one or more additional therapeutic agents.

In these and other embodiments, the treatment using Cabozantinib as a pharmaceutical formulation as described herein reaches one or more endpoints as defined by the RECIST criteria as described at www.irrecist.com/recist/recist-comparative/01.html (last visited Feb. 10, 2017, incorporated by reference in its entirety). The one or more endpoints are selected from the group consisting of objective response (OR), complete response (CR), partial response (PR), stable disease (SD), and progressive disease.

Cabozantininb can be administered as a CABOMETYX tablet. CABOMETYX is a tablet comprising Cabozantinib (S)-malate, microcrystalline cellulose, anhydrous lactose, hydroxypropyl cellulose, croscarmellose sodium, colloidal silicon dioxide magnesium stearate, and film coating comprising hypromellose, titanium dioxide, triacetin, and iron oxide yellow.

In an embodiment, the Cabozantinib (S)-malate is administered as a tablet formulation comprising approximately:
30-32 percent by weight of Cabozantinib, (S)-malate salt;
38-40 percent by weight of microcrystalline cellulose;
18-22 percent by weight of lactose;
2-4 percent by weight of hydroxypropyl cellulose;
4-8 percent by weight of croscarmellose sodium;
0.2-0.6 percent by weight of colloidal silicon dioxide;
0.5-1 percent by weight of magnesium stearate; and further comprising:

a film coating material comprising hypromellose, titanium dioxide, triacetin, and iron oxide yellow.

In another embodiment, the Cabozantinib (S)-malate is administered as a tablet formulation comprises approximately:
31-32 percent by weight of Cabozantinib, (S)-malate salt;
39-40 percent by weight of microcrystalline cellulose;
19-20 percent by weight of lactose;
2.5-3.5 percent by weight of hydroxypropyl cellulose;
5.5-6.5 percent by weight of croscarmellose sodium;
0.25-0.35 percent by weight of colloidal silicon dioxide;
0.7-0.8 percent by weight of magnesium stearate; and further comprising:
3.9-4.1 percent by weight of a film coating material comprising hypromellose, titanium dioxide, triacetin, and iron oxide yellow.

In another embodiment the Cabozantinib (S)-malate is administered as a tablet formulation selected from the group consisting of:

| | Theoretical Quantity (mg/unit dose) | | |
|---|---|---|---|
| | 20-mg | 40-mg | 60-mg |
| Ingredient | Tablet | Tablet | Tablet |
| Cabozantinib (S)-malate | 25.34 | 50.69 | 76.03 |
| Microcrystalline Cellulose, PH-102 | 31.08 | 62.16 | 93.24 |
| Lactose Anhydrous, 60M | 15.54 | 31.07 | 46.61 |
| Hydroxypropyl Cellulose, EXF | 2.400 | 4.800 | 7.200 |
| Croscarmellose Sodium | 4.800 | 9.600 | 14.40 |
| Colloidal Silicon Dioxide | 0.2400 | 0.4800 | 0.7200 |
| Magnesium Stearate (Non-Bovine) | 0.6000 | 1.200 | 1.800 |
| Opadry ® Yellow (03K92254) | 3.200 | 6.400 | 9.600 |
| Total tablet weight | 83.20 | 166.4 | 249.6 |

In another embodiment, the Cabozantinib (S)-malate is administered to treat urothelial cancer or another form of cancer as disclosed herein as a tablet formulation containing 20, 40, or 60 mg of Cabozantinib. 60 mg tablets are yellow film-coated, oval shaped with no score, debossed with “XL” on one side and “60” on the other side of the tablet; available in bottles of 30 tablets: NDC 42388-023-26. 40 mg tablets are yellow film-coated, triangle shaped with no score, debossed with “XL” on one side and “40” on the other side of the tablet; available in bottles of 30 tablets: NDC 42388-025-26. 20 mg tablets are yellow film-coated, round shaped with no score, debossed with “XL” on one side and “20” on the other side of the tablet; available in bottles of 30 tablets: NDC 42388-024-26.

In a further embodiment, the 60 mg of Cabozantinib is administered once daily as the CABOMETYX tablet formulation as described herein.

In another embodiment, the invention relates to the use of cabozantinib or a pharmaceutically acceptable salt thereof for the treatment of urothelial carcinoma or another form of cancer described herein. In one embodiment, the urothelial carcinoma is metastatic urothelial carcinoma is relapsed or refractory. In one embodiment, the Cabozantinib or pharmaceutically acceptable salt thereof is administered at a dose of 20-60 mg once daily. In another embodiment, the Cabozantinib or pharmaceutically acceptable salt thereof is administered at a dose of 20 mg once daily. In another embodiment, the Cabozantinib or pharmaceutically acceptable salt thereof is administered at a dose of 40 mg once daily. In another embodiment, the Cabozantinib or pharmaceutically acceptable salt thereof is administered at a dose of 60 mg once daily. In another embodiment, the Cabozantinib is administered as the (S)-malate. In another embodiment, the Cabozantinib is administered as Cabometyx tablets. In one embodiment, ORR was improved in patients with lung and/or liver metastases as compared to patients without lung and/or liver metastases.

In another embodiment, the invention relates to the treatment of urothelial carcinoma or another form of cancer described herein, comprising administering to a patient in need of such treatment, comprising administering a therapeutically effective amount of Cabozantinib or a pharmaceutically acceptable salt thereof. In one embodiment, the urothelial carcinoma is metastatic urothelial carcinoma is relapsed or refractory. In one embodiment, the Cabozantinib or pharmaceutically acceptable salt thereof is administered at a dose of 20-60 mg once daily. In another embodiment, the Cabozantinib or pharmaceutically acceptable salt thereof is administered at a dose of 20 mg once daily. In another embodiment, the Cabozantinib or pharmaceutically acceptable salt thereof is administered at a dose of 40 mg once daily. In another embodiment, the Cabozantinib or pharmaceutically acceptable salt thereof is administered at a dose of 60 mg once daily. In another embodiment, the Cabozantinib is administered as the (S)-malate. In another embodiment, the Cabozantinib is administered as Cabometyx tablets. In one embodiment, ORR was improved in patients with lung and/or liver metastases as compared to patients without lung and/or liver metastases.

In another embodiment, the invention relates to use of Cabozantinib or a pharmaceutically acceptable salt thereof in combination with nivolumab to treat urothelial carcinoma or another form of cancer as described herein. In one embodiment, the cabozantinib or pharmaceutically acceptable salt thereof is administered concurrently (at the same time) or sequentially (one after the other) with nivolumab. In one embodiment, the urothelial carcinoma is metastatic urothelial carcinoma. In one embodiment, the urothelial carcinoma is metastatic urothelial carcinoma is relapsed or refractory. In one embodiment, the Cabozantinib or a pharmaceutically acceptable salt thereof is administered at a dose of 20-60 mg once daily. In another embodiment, the Cabozantinib or pharmaceutically acceptable salt thereof is administered at a dose of 20 mg once daily. In another embodiment, the Cabozantinib or pharmaceutically acceptable salt thereof is administered at a dose of 40 mg once daily. In another embodiment, the Cabozantinib or pharmaceutically acceptable salt thereof is administered at a dose of 60 mg once daily. In another embodiment, the Cabozantinib is administered as the (S)-malate. In another embodiment, the Cabozantinib is administered as Cabometyx tablets.

In another embodiment, the invention relates to the treatment of urothelial carcinoma or another form of cancer described herein, comprising administering to a patient in need of such treatment, a therapeutically effective amount of Cabozantinib or a pharmaceutically acceptable salt thereof in combination with nivolumab. In one embodiment, the cabozantinib or pharmaceutically acceptable salt thereof is administered concurrently (at the same time) or sequentially (one after the other) with nivolumab. In one embodiment, the urothelial carcinoma is metastatic urothelial carcinoma. In one embodiment, the metastatic urothelial carcinoma is relapsed or refractory. In one embodiment, the Cabozantinib or a pharmaceutically acceptable salt thereof is administered at a dose of 20-60 mg once daily. In another embodiment, the Cabozantinib or pharmaceutically acceptable salt thereof is administered at a dose of 20 mg once daily. In another embodiment, the Cabozantinib or pharmaceutically acceptable salt thereof is administered at a dose of 40 mg once daily. In another embodiment, the Cabozantinib or pharmaceutically acceptable salt thereof is administered at a dose of 60 mg once daily. In another embodiment, the Cabozantinib is administered as the (S)-malate. In another embodiment, the Cabozantinib is administered as Cabometyx tablets.

In another embodiment, the Cabozantinib or a pharmaceutically acceptable salt thereof is administered to patients once daily and nivolumab is administered intravenously every two weeks. In one embodiment, cabozantinib or a pharmaceutically acceptable salt thereof is administered concurrently (at the same time) or sequentially (one after the other) with nivolumab. In one embodiment, 20 mg of Cabozantinib or a pharmaceutically acceptable salt thereof is administered once daily for a 28 day cycle and nivolumab is administered 1 mg/kg IV on days 1 and 14 of the 28 day cycle. In one embodiment, 40 mg of Cabozantinib or a pharmaceutically acceptable salt thereof is administered once daily for a 28 day cycle and nivolumab is administered 1 mg/kg IV on days 1 and 14 of the 28 day cycle. In one embodiment, 40 mg of Cabozantinib or a pharmaceutically acceptable salt thereof is administered once daily for a 28 day cycle and nivolumab is administered 3 mg/kg IV on days 1 and 14 of the 28 day cycle. In one embodiment, 60 mg of Cabozantinib or a pharmaceutically acceptable salt thereof is administered once daily for a 28 day cycle and nivolumab is administered 1 mg/kg IV on days 1 and 14 of the 28 day cycle. In one embodiment, 60 mg of Cabozantinib or a pharmaceutically acceptable salt thereof is administered once daily for a 28 day cycle and nivolumab is administered 3 mg/kg IV on days 1 and 14 of the 28 day cycle.

In another embodiment of the treatment method, the dosing schedule is described in the following table.

| | | | |
|---|---|---|---|
| Dose | | | |
| Level | Cabozantinib | Nivolumab | Ipilimumab x 4 doses |

PART I

| | | | | |
|---|---|---|---|---|
| 1 | 40 | mg po daily | 1 mg/kg q 2 wks | 0 |
| 2 | 40 | mg po daily | 3 mg/kg q 2 wks | 0 |
| 3 | 60 | mg po daily | 1 mg/kg q 2 wks | 0 |
| 4 | 60 | mg po daily | 3 mg/kg q 2 wks | 0 |

PART II

| | | | | |
|---|---|---|---|---|
| 5 | 40 | mg po daily | 1 mg/kg q 3 wks | 1 mg/kg q 3 wks |
| 6 | 40 | mg po daily | 3 mg/kg q 3 wks | 1 mg/kg q 3 wks |
| 7 | 60 | mg po qd | 3 mg/kg q 3 wks | 1 mg/kg q 3 wks |

In a further embodiment, the treatment method has two parts. In the first part, 40 mg of Cabozantinib or a pharmaceutically acceptable salt thereof is administered once daily and Nivolumab is administered 1 mg/kg every two weeks. In one embodiment of this part, cabozantinib or a pharmaceutically acceptable salt thereof is administered concurrently (at the same time) or sequentially (one after the other) with nivolumab. Alternatively, in a further embodiment of the first part, 40 mg of Cabozantinib or a pharmaceutically acceptable salt thereof is administered once daily and Nivolumab is administered 3 mg/kg every two weeks. In one embodiment of this alternative, cabozantinib or a pharmaceutically acceptable salt thereof is administered concurrently (at the same time) or sequentially (one after the other) with nivolumab. Alternatively, in a further embodiment of the first part, 60 mg of Cabozantinib or a pharmaceutically acceptable salt thereof is administered once daily and Nivolumab is administered 1 mg/kg every two weeks. In one embodiment of this alternative, cabozantinib or a pharmaceutically acceptable salt thereof is administered concurrently (at the same time) or sequentially (one after the other) with nivolumab. Alternatively, in a further embodiment of the first part, 60 mg of Cabozantinib or a pharmaceutically acceptable salt thereof is administered once daily and Nivolumab is administered 3 mg/kg every two weeks. In one embodiment of this alternative, cabozantinib or a pharmaceutically acceptable salt thereof is administered concurrently (at the same time) or sequentially (one after the other) with nivolumab.

In the second part, 40 mg of Cabozantinib or a pharmaceutically acceptable salt thereof is administered once daily, Nivolumab is administered 1 mg/kg every two weeks, and Ipilimumab is administered 1 mg/kg every three weeks. In one embodiment of this part, cabozantinib or a pharmaceutically acceptable salt thereof is administered concurrently (at the same time) or sequentially (one after the other) with nivolumab and ipilimumab. Alternatively, in a further embodiment of the first part, 40 mg of Cabozantinib or a pharmaceutically acceptable salt thereof is administered once daily, Nivolumab is administered 3 mg/kg every two weeks, and Ipilimumab is administered 1 mg/kg every three weeks. In one embodiment of this alternative, cabozantinib or a pharmaceutically acceptable salt thereof is administered concurrently (at the same time) or sequentially (one after the other) with nivolumab and ipilimumab. Alternatively, in a further embodiment of the first part, 60 mg of Cabozantinib or a pharmaceutically acceptable salt thereof is administered once daily Nivolumab is administered 1 mg/kg every two weeks, and Ipilimumab is administered 1 mg/kg every three weeks. In one embodiment of this alternative, cabozantinib or a pharmaceutically acceptable salt thereof is administered concurrently (at the same time) or sequentially (one after the other) with nivolumab and ipilimumab. Alternatively, in a further embodiment of the first part, 60 mg of Cabozantinib or a pharmaceutically acceptable salt thereof is administered once daily and Nivolumab is administered 3 mg/kg every two weeks, and Ipilimumab is administered 1 mg/kg every three weeks. In one embodiment of this alternative, cabozantinib or a pharmaceutically acceptable salt thereof is administered concurrently (at the same time) or sequentially (one after the other) with nivolumab and ipilimumab.

In the above methods and uses, Cabozantinib can be administered as a malate salt. In one embodiment, Cabozantinib is administered as the (S)-malate salt.

### Examples

This example provides results of a Phase I study of Cabozantinib (Cabo)+Nivolumab (Nivo) and (CaboNivo) Cabo+Nivolumab+Ipilimumab (Ipi) (CaboNivoIpi) in patients with metastatic urothelial carcinoma and other genitourinary malignancies.

Cabozantinib is primarily VEGFR2 and MET pathways inhibitor. It has shown clinical activity in pretreated patients with relapsed/refractory metastatic urothelial carcinoma (mUC).

Nivolumab is sold under the name OPDIVO. It has previously been approved for the treatment of unresectable or metastatic melanoma and disease progression following ipilimumab and, if BRAF V600 mutation positive, a BRAF inhibitor. Nivolumab is administered at a dose 3 mg/kg as an intravenous infusion over 60 minutes every 2 weeks. (2.1) (See www.accessdata.fda.gov/drugsatfda_docs/label/2014/1255541 b1.pdf last visited Feb. 9, 2017). Nivolumab has clinical activity in patients with metastatic urothelial carcinoma (mUC) who have progressed despite prior platinum-containing chemotherapy (Phase II CheckMate 125 study). Five PD-1-PD-L1 inhibitors received accelerated approvals in US based on response rates.

Ipilimumab is sold under the name YERVOY. It has previously been approved for the treatment of unresectable or metastatic melanoma. Ipilimumab is administered at a dose of 3 mg/kg administered intravenously over 90 minutes every 3 weeks for a total of 4 doses. (See packageinserts.bms.com/pi/piyervoy.pdf last visited Feb. 9, 2017).

### Combination of Nivolumab+Ipilimumab

The combination of nivolumab plus ipilimumab has shown manageable toxicity and clinical activity in mUC (Phase I/II CheckMate 032 study)

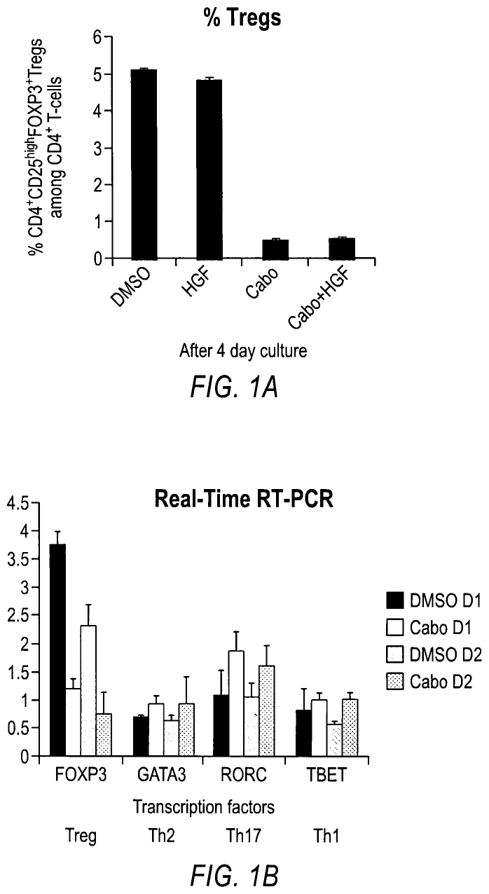

As shown in FIG. 1, cabozantinib appears to decrease the Treg population in urothelial cancer patients, by: 1. Directly killing the tregs, and 2. Influencing treg polarization. See Apolo, A B, et al., J. Clin. Oncol. 2014; 32s:abstr 4501.

With reference to FIGS. 1A and 1B, peripheral blood mononuclear cells (PBMC) were obtained by centrifugation from buffy coat. PBMCs were incubated for 3-4 days in a 24 well plate with 10% FBS-RPMI medium with DMSO (control) or Cabozantinib (10 uM). Representative data obtained from three different donors were shown. Cabozantinib suppresses only Foxp3 among master regulator transcription factors of CD4+ T-cell polarization. Naïve CD4+ T-cells can polarize toward four CD4+ lineages.

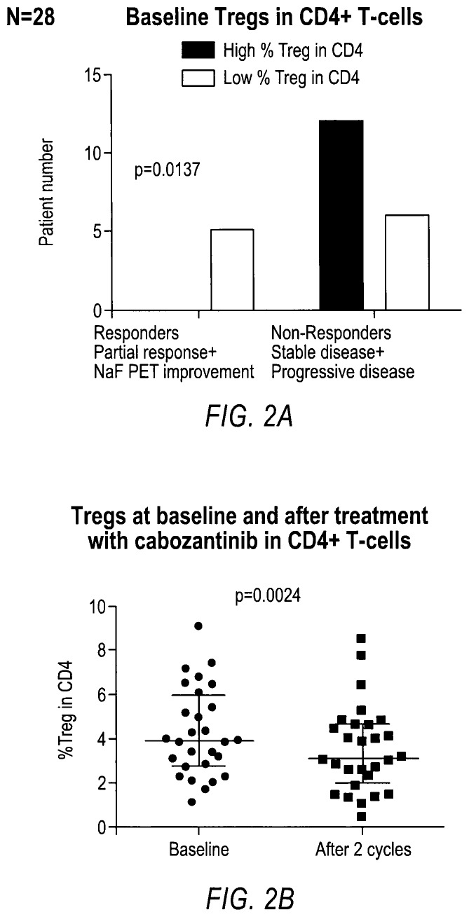

With reference to FIG. 2, it is believed that cabozantinib downregulates regulatory T cell population by acting on T cell polarization via inhibition of transcription factor Foxp3. The biologic rationale for combining cabozantinib and immune checkpoint blockade in mUC can be extrapolated from the results shown in FIGS. 2A and 2B. In FIG. 2A, patients with lower percent Treg (CD4+CD25hiFoxp3+) in CD4+ T-cells (<3.89%=median value) at baseline had better response compared with those who had higher percent Treg at baseline. As shown in FIG. 2B, the percent Treg among CD4+ T-cells decreased significantly after treatment with Cabozantinib.

Study

The study provides results of a Phase I study of Cabozantinib+Nivolumab and Cabozantinib+Nivolumab+Ipilimumab in patients with metastatic urothelial carcinoma and other genitourinary malignancies. The purpose of the study was to determine the dose limiting toxicity (DLT) and recommended Phase II dose (RP2D) of the combination of CaboNivo and separately the combination of CaboNivolpi. In the study, DLT was defined as any event requiring a patient to discontinue study treatment. The DLT evaluation period was 4 weeks (Part 1) and 6 weeks (Part 2).

Secondary endpoints for the study included overall response rate (ORR) by RECIST v1.1; duration of response; progression free survival (PFS); and overall survival (OS). Exploratory Endpoints included the identification of immune subset biomarkers associated with PD-L1 and MET expression as well as correlation with efficacy.

Key eligibility criteria for the study included:
Histologically confirmed diagnostic of genitourinary (GU) malignancy;
Patients with metastatic GU malignancy who has disease progression on at least one standard therapy or there must be no standard treatment that prolongs survival;
A Karnofsky performance status of greater than or equal to 70 percent; and
One evaluable site of disease or bone disease by NaF PET/CT.

Patients (42) involved in the study were determined to have baseline characteristics as shown in Table 3.

TABLE 3

| Characteristic | N = 42 | |
|---|---|---|
| Median age, years (range) | 56 | (31-77) |
| Male, % (N) | 90 | (38) |
| No. of prior systemic | | |
| regimens, % (N) | | |
| 0 | 14 | (6) |
| 1 | 19 | (8) |
| >2 | 67 | (28) |
| KPS, % (N) | | |
| 70% | 2 | (1) |
| 80% | 36 | (15) |
| 90% | 62 | (26) |
| Baseline No. of metastatic | | |
| sites, % (N) | | |
| 1 | 17 | (7) |
| 2 | 33 | (14) |
| 3 | 50 | (21) |
| Type of Tumor % | | |
| Urothelial Carcinoma of the | 29 | (12) |
| bladder | | |
| Non-UC GU malignancies | 71 | (30) |

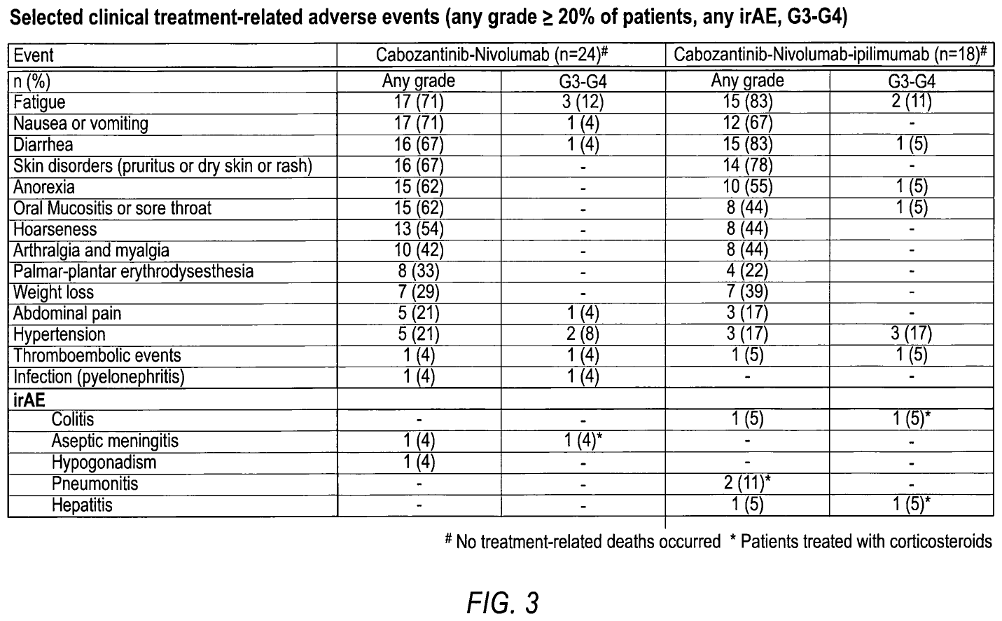

Clinical treatment-adverse events reported during the study are shown in FIG. 3. Notably, no treatment related deaths were observed.

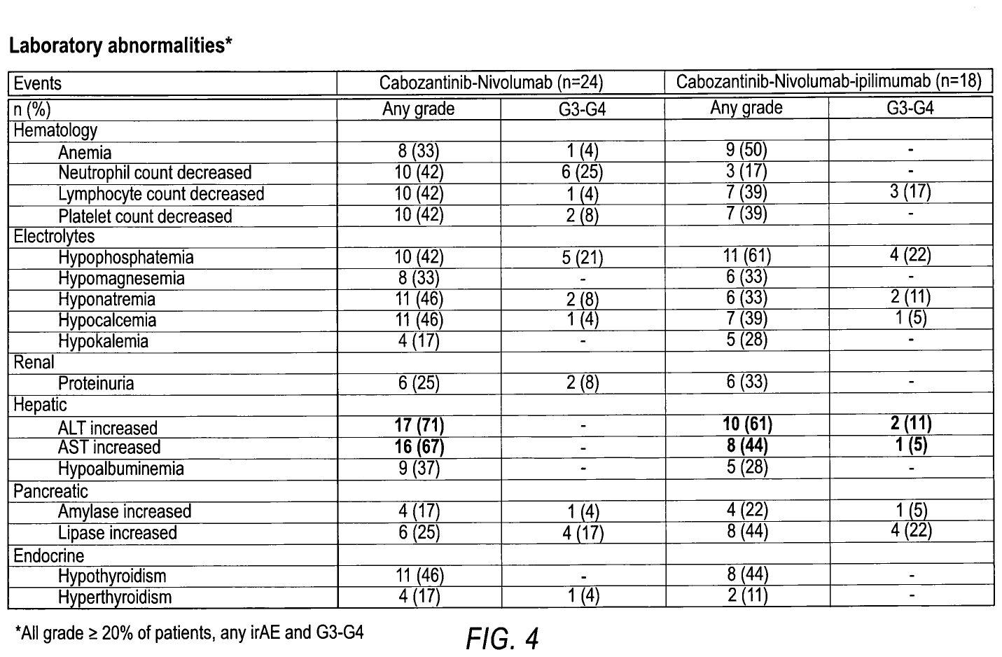

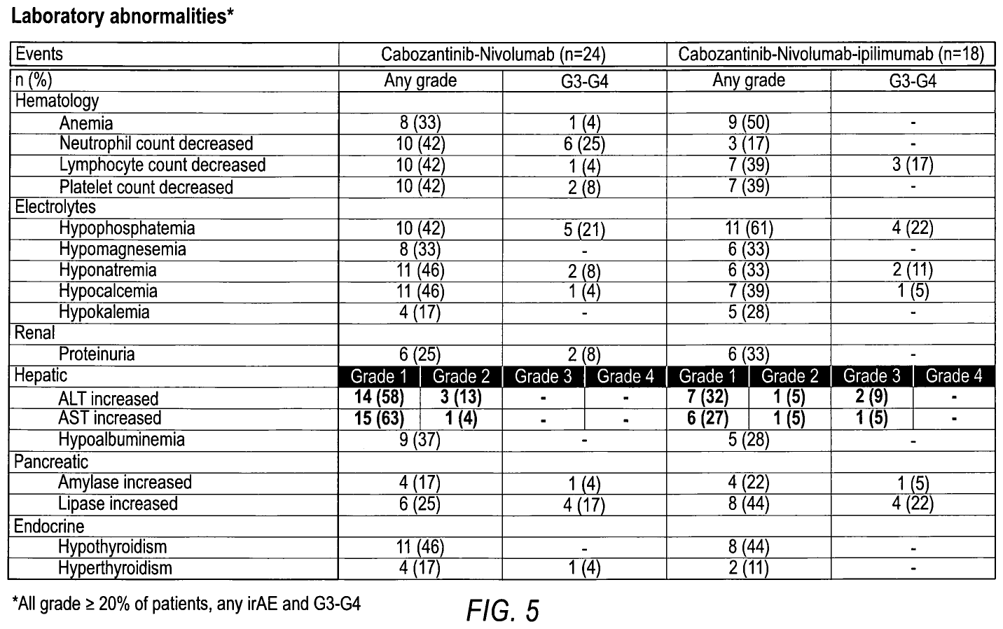

Determination of laboratory abnormality events reported are provided in FIGS. 4 and 5.

Transaminase elevation criteria and liver function management/dosing decisions were reported and are summarized in Tables 4 and 5.

TABLE 4
Transaminase Elevations

| | |
|---|---|
| Transaminase elevation | |
| CTCAE v4.0 | Intervention |

Subjects with AST and ALT less than or equal to the ULN at baseline

| | |
|---|---|
| Grade 1 | Continue cabozantinib |
| Grade 2 | Continue cabozantinib with at least weekly monitoring of LFTs for 2 weeks. |
| Grade 3 | Interrupt cabozantinib treatment and monitor with at least twice weekly |
| | LFTs until Grade ≤2. Resume cabozantinib at same dose level or at one |
| | dose-level reduction at the discretion of investigator continue with at least |
| | weekly LFTs until resolution to Grade ≤1. |
| Grade 4 | Discontinue study treatment permanently. LFTs should be monitored as |
| | clinically indicated, at least 2-3 times per week, until resolution to |
| | Grade ≤1. If the subject was unequivocally deriving clinical benefit, the |
| | subject may be able to resume treatment at a lower dose of cabozantinib as |
| | determined by the investigator and sponsor but only with sponsor approval. |

TABLE 5
Liver Function

| Liver Function AST, | Management/Next Dose for Nivolumab and combination |
|---|---|
| ALT, Bilirubin | Nivolumab/Ipilimumab |
| ≤Grade 1 | Continue at current dose at investigator discretion. |
| Grade 2 | If normal LFTs at baseline, then hold until grade 1 or baseline. If grade 1 at |
| | baseline and asymptomatic, continue nivolumab or nivolmnab/ipilimumab and |
| | increase to at least weekly monitoring of LFTs for 2 weeks. Resume at same |
| | dose level. |
| Grade 3 | Interrupt treatment of nivolumab or nivolumab/ipilimumab and monitor with at |
| | least weekly LFTs until Grade ≤2. Resume nivolumab or ipilimumab at the |
| | same dose level and then continue with at least weekly LFTs until resolution to |
| | Grade ≤1. If no improvement or worsening by 4 weeks initiate steroid |
| | treatment. |
| Grade 4 | Off protocol therapy |

Continued treatment of active immune mediated hepatitis may exacerbate ongoing inflammation. Holding drug to evaluate LFT changes and early treatment are recommended.
LFT changes may occur during steroid tapers from other events and may occur together with other GI events including cholecystitis/pancreatitis.
Nivolumab and ipilimumab may be held and steroids initiated at any time if clinically indicated.

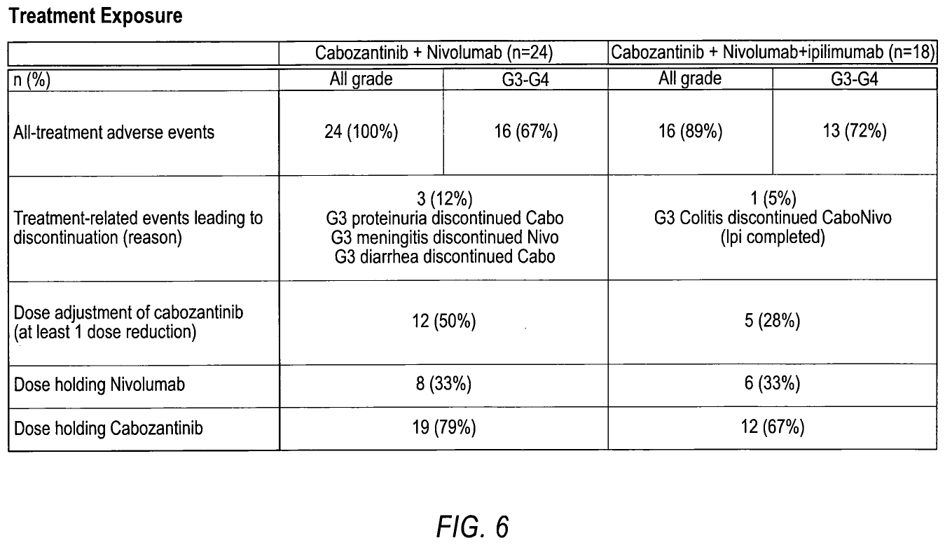

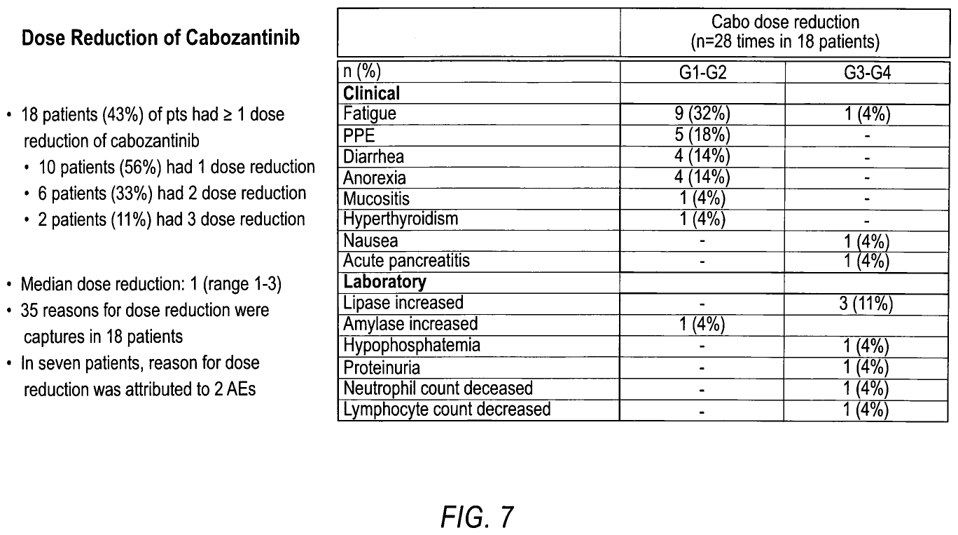

Other treatment exposure events, dose discontinuation or dose adjustments were reported as shown in FIGS. 6 and 7.

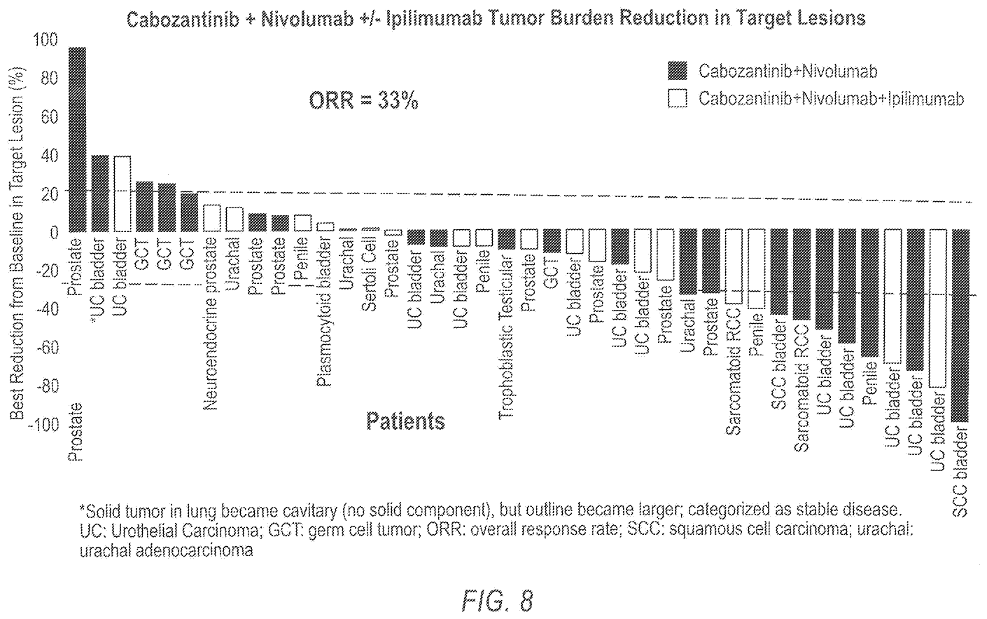

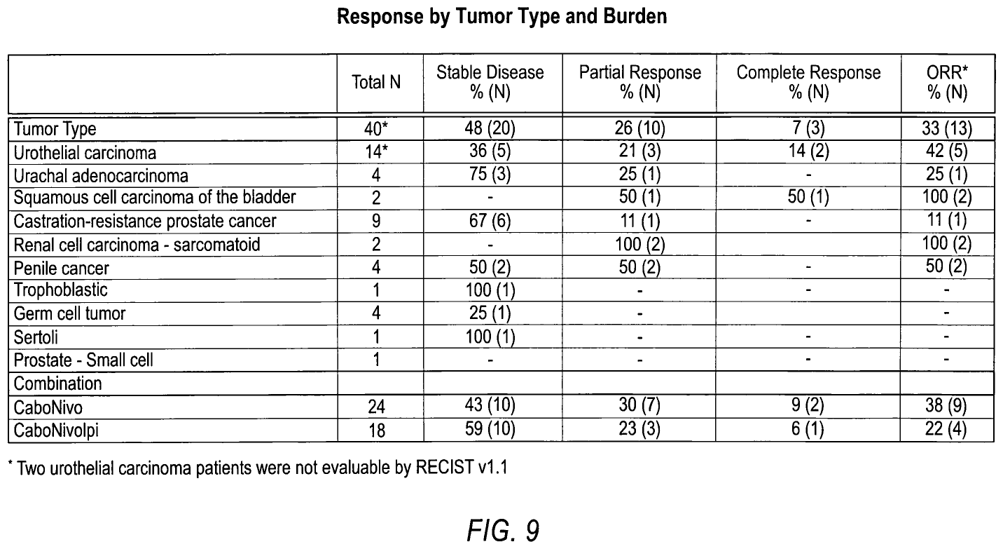

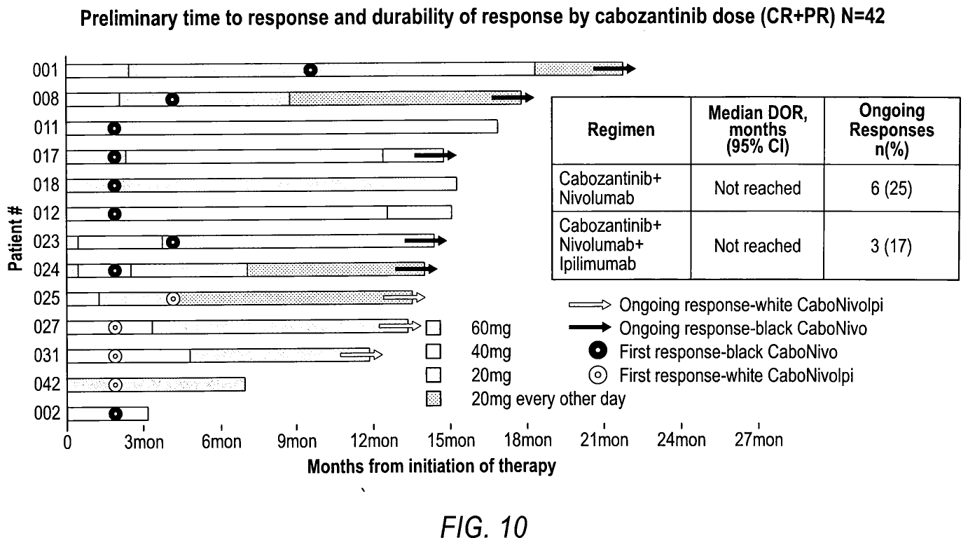

As shown in FIG. 8, the combination of Cabozantinib+Nivolumab+/−Ipilimumab was found to reduce the tumor burden in patient target lesions. Further analysis of the effects of the combination of Cabozantinib+Nivolumab+/−Ipilimumab on various tumor types and by regimen was determined. As shown in FIG. 9, the combination of Cabozantinib+Nivolumab (CaboNivo) and Cabozantinib+Nivolumab+Ipilimumab (CaboNivolpi) was tested against various tumor types. In addition, preliminary time to response and durability of response by cabozantinib dose (CR+PR) was measured as shown in FIG. 10.

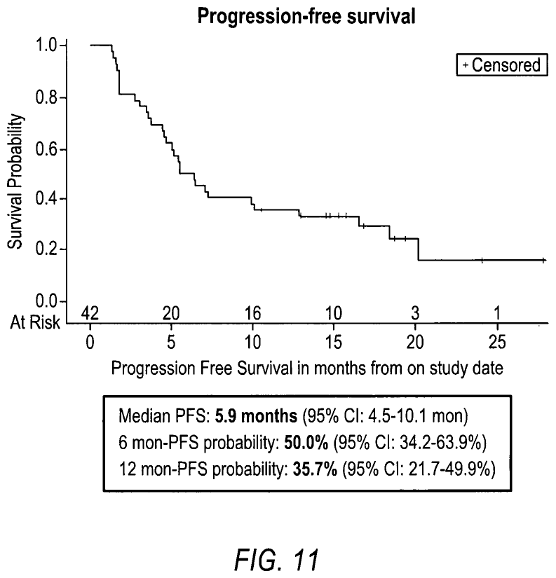

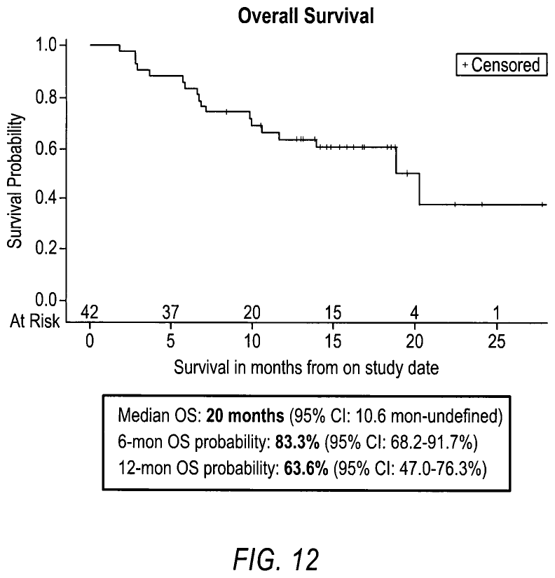

Time event outcomes for patients including progression free survival and overall survival are shown in FIGS. 11 and 12. The median progression free survival was found to be 5.9 months and a median overall survival of 20 months. The median potential follow-up was determined to be 16.0 months.

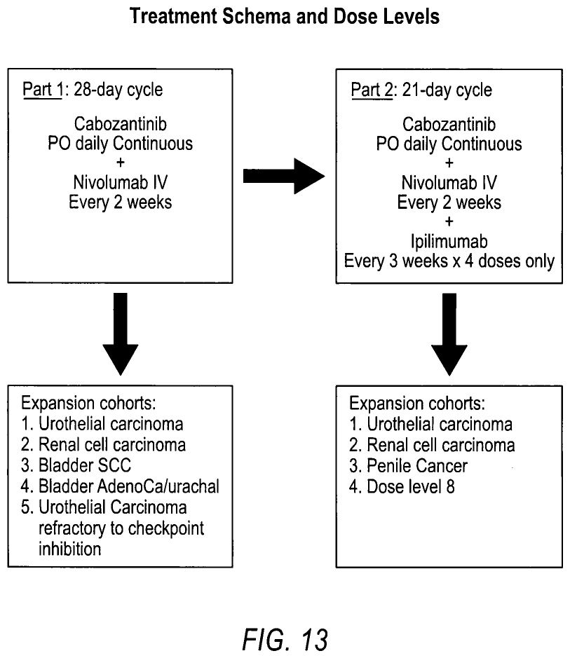

FIG. 13 depicts the study design for an expansion study of cabozantinib plus nivolumab (CaboNivo) alone or with Ipilimumab (CaboNivolpi) in patients with metastatic urothelial carcinoma and other genitourinary tumors.

Dose Reduction

18 patients (43%) had ≥1 dose reduction of cabozantinib.

10 patients (56%) had 1 dose reduction.

6 patients (33%) had 2 dose reductions.

2 patients (11%) had 3 dose reductions.

Median dose reduction: 1 (range 1-3).

35 reasons for dose reduction were captured in 18 patients.

In seven patients, the reason for dose reduction was attributed to 2 AEs.

### CONCLUSIONS

Both the combination of Cabozantinib and Nivolumab and the combination of Cabozantinib, Nivolumb, and Ipilimumab are safe and well-tolerated

The recommended phase II dose for CaboNivo is cabozantinib 40 mg+nivolumab 3 mg/kg.

The recommended phase II dose for CaboNivolpi is cabozantinib 40 mg+nivolumab 3 mg/kg+ipilimumab 1 mg/kg.

Both the combination of Cabozantinib and Nivolumab and the combination of Cabozantinib, Nivolumb, and Ipilimumab are active in genitourinary tumors, particularly urothelial carcinoma.

Rare tumors such as squamous cell carcinoma of the bladder, urachal adenocarcinoma, and penile cancer demonstrated response to the combination.

Larger cohorts of metastatic urothelial carcinoma and rare genitourinary tumors are ongoing.

### OTHER EMBODIMENTS

The foregoing disclosure has been described in some detail by way of illustration and example, for purposes of clarity and understanding. The invention has been described with reference to various specific and preferred embodiments and techniques. However, it should be understood that many variations and modifications can be made while remaining within the spirit and scope of the invention. It will be obvious to one of skill in the art that changes and modifications can be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

1. A method of treating a cancer selected from the group consisting of urothelial carcinoma, metastatic urothelial carcinoma, urachal adenocarcinoma, squamous cell carcinoma of the bladder, squamous cell carcinoma, urachal adenocarcinoma, castration-resistant prostate cancer, renal cell carcinoma-sarcomatoid tumor, trophoblastic tumor, germ cell cancer, and penile cancer, comprising administering to a patient in need of such treatment Cabozantinib, or a pharmaceutically acceptable salt thereof, in combination with one or more therapeutic agents selected from the group consisting of an anti-CTLA4 monoclonal antibody or an IgG4 anti-PD-1 monoclonal antibody or both an anti-CTLA4 monoclonal antibody and an IgG4 anti-PD-1; wherein the structure of Cabozantinib is:

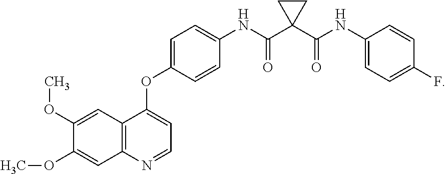 [SMILES: COc1cc2nccc(Oc3ccc(NC(=O)C4(C(=O)Nc5ccc(F)cc5)CC4)cc3)c2cc1OC]

2. The method of claim 1, wherein the cancer is selected from urothelial cancer, squamous cell carcinoma of the bladder, squamous cell carcinoma, urachal adenocarcinoma, and penile cancer.

3. The method of claims 1-2, wherein the cancer is metastatic urothelial carcinoma.

4. The method of claims 1-3, wherein the Cabozantinib or pharmaceutically acceptable salt thereof is administered as a malate salt.

5. The method of claims 1-4, wherein the Cabozantinib or pharmaceutically acceptable salt thereof is administered as the (S)-malate salt.

6. The method of claims 1-5, wherein Cabozantinib (S)-malate is administered as a tablet comprising Cabozantinib (S)-malate, microcrystalline cellulose, anhydrous lactose, hydroxypropyl cellulose, croscarmellose sodium, colloidal silicon dioxide magnesium stearate, and film coating comprising hypromellose, titanium dioxide, triacetin, and iron oxide yellow.

7. The method of claims 1-6, wherein the Cabozantinib (S)-malate is administered as a tablet formulation comprising approximately:
30-32 percent by weight of Cabozantinib, (S)-malate salt;
38-40 percent by weight of microcrystalline cellulose;
18-22 percent by weight of lactose;
2-4 percent by weight of hydroxypropyl cellulose;
4-8 percent by weight of croscarmellose sodium;
0.2-0.6 percent by weight of colloidal silicon dioxide;
0.5-1 percent by weight of magnesium stearate; and further comprising:
a film coating material comprising hypromellose, titanium dioxide, triacetin, and iron oxide yellow.

8. The method of claims 1-7, wherein the Cabozantinib (S)-malate is administered as a tablet formulation comprising approximately (% w/w):
31-32 percent by weight of Cabozantinib, (S)-malate salt;
39-40 percent by weight of microcrystalline cellulose;
19-20 percent by weight of lactose;
2.5-3.5 percent by weight of hydroxypropyl cellulose;
5.5-6.5 percent by weight of croscarmellose sodium;
0.25-0.35 percent by weight of colloidal silicon dioxide;
0.7-0.8 percent by weight of magnesium stearate; and further comprising:
3.9-4.1 percent by weight of a film coating material comprising hypromellose, titanium dioxide, triacetin, and iron oxide yellow.

9. The method of claims 1-8, wherein the Cabozantinib or pharmaceutically acceptable salt thereof is administered as a tablet formulation containing 20, 40, or 60 mg of Cabozantinib.

10. The method of claims 1-8, wherein the Cabozantinib or pharmaceutically acceptable salt thereof is administered as a tablet formulation containing 20 mg of Cabozantinib.

11. The method of claims 1-8, wherein the Cabozantinib or pharmaceutically acceptable salt thereof is administered as a tablet formulation containing 40 mg of Cabozantinib.

12. The method of claims 1-8, wherein the Cabozantinib or pharmaceutically acceptable salt thereof is administered as a tablet formulation containing 60 mg of Cabozantinib.

13. The method of claims 1-12, wherein the anti-CTLA4 monoclonal antibody is nivolumab.

14. The method of claims 1-13, wherein the IgG4 anti-PD-1 monoclonal antibody is ipilimumab.

15. The method of claims 1-14 wherein the dose of cabozantinib or a pharmaceutically acceptable salt thereof is 40 mg once daily and the dose of nivolumab is 3 mg/kg once daily.

16. The method of claims 1-14 wherein the dose of cabozantinib or a pharmaceutically acceptable salt thereof is 40 mg once daily, the dose of nivolumab is 3 mg/kg once daily, and the dose of ipilimumab is 1 mg/kg once daily.

17. The method of claims 1-16, wherein the patient has undergone prior cancer treatment.

18. The use of cabozantinib or a pharmaceutically acceptable salt thereof for the treatment of a cancer selected from the group consisting of urothelial carcinoma, metastatic urothelial carcinoma, urachal adenocarcinoma, bladder squamous cell carcinoma of the bladder, castration-resistant prostate cancer, renal cell carcinoma-sarcomatoid tumor, trophoblastic tumor, germ cell cancer, and penile cancer.

19. The use of a combination of cabozantinib or a pharmaceutically acceptable salt thereof and nivolumab for the treatment of a cancer selected from group consisting of urothelial carcinoma, metastatic urothelial carcinoma, urachal adenocarcinoma, bladder squamous cell carcinoma of the bladder, castration-resistant prostate cancer, renal cell carcinoma-sarcomatoid tumor, trophoblastic tumor, germ cell cancer, and penile cancer.

20. The use of a combination of cabozantinib or a pharmaceutically acceptable salt thereof and nivolumab and ipilimumab for the treatment of a cancer selected from group consisting of urothelial carcinoma, metastatic urothelial carcinoma, urachal adenocarcinoma, bladder squamous cell carcinoma of the bladder, castration-resistant prostate cancer, renal cell carcinoma-sarcomatoid tumor, trophoblastic tumor, germ cell cancer, and penile cancer.

21. The uses of claims 18-20, wherein Cabozantinib or a pharmaceutically acceptable salt thereof is administered as a tablet formulation containing 20, 40, or 60 mg of Cabozantinib.

22. The uses of claims 18-20, wherein Cabozantinib or a pharmaceutically acceptable salt thereof is administered as a tablet formulation containing 20 mg of Cabozantinib.

23. The uses of claims 18-20, wherein Cabozantinib or a pharmaceutically acceptable salt thereof is administered as a tablet formulation containing 40 mg of Cabozantinib.

24. The uses of claims 18-20, wherein Cabozantinib or a pharmaceutically acceptable salt thereof is administered as a tablet formulation containing 60 mg of Cabozantinib.

25. The uses of claims 19-20, wherein the dose of Cabozantinib or a pharmaceutically acceptable salt thereof is 40 mg once daily and the dose of Nivolumab is 3 mg/kg once daily.

26. The uses of claims 19-20, wherein the dose of Cabozantinib or a pharmaceutically acceptable salt thereof is 40 mg once daily, the dose of Nivolumab is 3 mg/kg once daily, and the dose of Ipilimumab is 1 mg/kg once daily.

27. The uses of claims 18-26, wherein cabozantinib is administered as a malate salt.

28. The uses of claims 18-26, wherein cabozantinib is administered as the (S)-malate salt.
